(19) United States
(12) Patent Application Publication
(10) Pub. No.: US 2024/0246052 A1
(43) Pub. Date: Jul. 25, 2024

(54) FLUIDIZED BED REACTOR, AND DEVICE FOR PREPARING LOW-CARBON OLEFIN AND METHOD FOR PREPARING LOW-CARBON OLEFIN

(71) Applicants: CHINA PETROLEUM & CHEMICAL CORPORATION, Beijing (CN); SHANGHAI RESEARCH INSTITUTE OF PETROCHEMICAL TECHNOLOGY, SINOPEC, Shanghai (CN)

(72) Inventors: Hongyuan ZONG, Shanghai (CN); Guozhen QI, Shanghai (CN); Pan GAO, Shanghai (CN); Xiaohong LI, Shanghai (CN); Jing CAO, Shanghai (CN); Zhinan YU, Shanghai (CN); Yanxue WANG, Shanghai (CN); Fei PENG, Shanghai (CN)

(21) Appl. No.: 18/566,128

(22) PCT Filed: Jun. 23, 2022
(86) PCT No.: PCT/CN2022/100672

§ 371 Date: Dec. 1, 2023

(30) Foreign Application Priority Data
Jun. 23, 2021 (CN) 202110697978.8

(51) Int. Cl. B01J 8/18; B01J 8/00; B01J 8/26; C07C 1/24

(52) U.S. Cl. CPC B01J 8/1827; B01J 8/0055; B01J 8/1863; B01J 8/26; C07C 1/24; B01J 2208/00752; B01J 2208/00849; B01J 2208/00902; B01J 2208/00938; C07C 2529/40

(57) ABSTRACT

A fluidized bed reactor, a device for preparing low-carbon olefin, and a method for preparing low-carbon olefin are provided. The reaction zone of the fluidized bed reactor is sequentially, from bottom to top, provided with a raw material first distributor, a raw material second distributor and a catalyst distributor; the catalyst distributor is connected with the catalyst second feeding inlet; a dense-phase zone is formed between the raw material first distributor and the raw material second distributor, and the area where the catalyst distributor is located is formed as a catalyst distribution zone connected with the dense-phase zone; and at least one catalyst first feeding inlet(s) is provided on the side wall of reactor of the dense-phase zone. The distribution of the catalyst and the full contact of the catalyst with the raw materials can be achieved using the fluidized bed reactor and the device.

### TECHNICAL FIELD

The present invention relates to the field of olefin preparation, specifically, to a fluidized bed reactor, a device for preparing low-carbon olefin, and a method for preparing low-carbon olefin.

### BACKGROUND ART

Low-carbon olefin, namely ethylene and propylene, are two important basic chemical raw materials, and their demand is constantly increasing. Generally, ethylene and propylene are produced through petroleum routes, however, due to the limited supply and high price of petroleum resources, the cost of producing ethylene and propylene from petroleum resources continues to increase. In recent years, people have begun to vigorously develop technologies for converting alternative raw materials into ethylene and propylene. Wherein, an important kind of alternative raw materials for the production of low-carbon olefin is oxygen-containing compounds, such as alcohols (methanol, ethanol), ethers (dimethyl ether, methyl ethyl ether), esters (dimethyl carbonate, methyl formate), etc. These oxygen-containing compounds can be converted from coal, natural gas, biomass and other energy sources. Certain oxygen-containing compounds can already be produced on a large scale, such as methanol, which can be produced from coal or natural gas, and the process is very mature and can achieve a production scale of millions of tons. Due to the wide range of sources of oxygen-containing compounds and the economy of the process of converting them to low-carbon olefin, the process of converting oxygen-containing compounds to olefin (OTO), especially the process of converting methanol to olefin (MTO), is receiving more and more attention.

The current device for producing olefin from oxygen-containing compounds is similar to the catalytic cracking unit, both of which adopt a continuous reaction-regeneration mode. PCT application WO 2018072139A1 discloses a turbulent fluidized bed reactor, device and method for preparing propylene and C4 hydrocarbons from oxygen-containing compounds; this technical solution sets n reactor feed distributors in the reaction zone of the turbulent fluidized bed reactor, the concentration of oxygen-containing compounds is relatively uniform, which weakens the inhibition of the olefin alkylation reaction by the MTO reaction, and the regenerated catalyst is directly introduced at the bottom of the reaction zone, which is beneficial to the alkylation reactions of ethylene, propylene and methanol.

Chinese patent applications with publication numbers CN108794294A and CN108786669A respectively record a fluidized bed distributor and a fluidized bed reactor including the fluidized bed distributor, wherein the fluidized bed distributor includes a first distributor and a second distributor, the first distributor is located at the bottom of the fluidized bed, and the second distributor is located in at least one area downstream of the gas flow of the first distributor, and the feed is distributed in different areas through different raw materials streams to achieve mass transfer control, then coordinate and optimize the co-feeding system.

The Chinese patent application with publication number CN107235821A describes a device for producing olefin from methanol, wherein a first external circulation catalyst distributor and a first catalyst redistributor are provided in the reaction zone of the fluidized bed reactor, and a second external circulation catalyst distributor, a low activity catalyst distributor, the cooled catalyst distributor and the second catalyst redistributor are provided in the regeneration zone, which not only ensure the uniform distribution of temperature and activity of the reactants and catalysts, but also ensure the contact effect between the reaction gas and the catalyst.

In the prior art, there is still a phenomenon of uneven distribution of the regenerated catalyst in the fluidized bed reactor, resulting in large bed temperature fluctuations and a greater impact on the selection of dienes; in addition, the regeneration process mainly uses air as the regeneration gas. By adjusting the amount of auxiliary gas in the regeneration feed gas, the “temperature flying” phenomenon during the regeneration process can be prevented. However, this method will produce a large amount of greenhouse gas CO₂ and is not conducive to environmental protection. If air is used to burn carbon deposit to partially regenerate the catalyst, the rate of burning carbon is faster, which is not conducive to the control of the amount of residual carbon in the catalyst and increases the difficulty in the operation process.

### SUMMARY OF THE INVENTION

The object of the present invention is to overcome the problems of uneven catalyst distribution, insufficient regeneration heat utilization and low yield of low-carbon olefin in the existing devices for preparing low-carbon olefin, and to provide a fluidized bed reactor and a device for preparing low-carbon olefin and a method for preparing low-carbon olefin.

In the first aspect, the invention provides a fluidized bed reactor, which includes:
in the reaction zone of the fluidized bed reactor, a raw material first distributor, a raw material second distributor and a catalyst distributor, which are arranged in sequence from bottom to top, which are used to distribute the gaseous raw material(s), wherein the raw material first distributor is below the raw material second distributor; wherein the raw material first distributor and the raw material second distributor have the same or different opening ratios, each independently being 0.05-5%;
a raw material first inlet at the bottom of the fluidized bed reactor, which allows at least a part of the raw material(s) to be distributed twice sequentially through the raw material first distributor and the raw material second distributor;
the catalyst distributor includes a main guide pipe of the catalyst distributor, which is coaxially distributed with the raw material first distributor and the raw material second distributor, wherein the catalyst distributor main guide pipe passes through the raw material first distributor and the raw material second distributor from bottom to top.

In the second aspect, the present invention provides a device for preparing low-carbon olefin, wherein the device includes a fluidized bed reactor according to the invention, a settler and a regenerator, and the side wall of the reactor is provided with at least one catalyst first feeding inlet(s) for feeding the first catalyst to the location between the raw material first distributor and the raw material second distributor, and the bottom of the reactor is provided with a catalyst second feeding inlet(s) for feeding the second catalyst feed into the main guide pipe of the distributor; wherein: the settler is connected with the upper part of the reaction zone of the fluidized bed reactor, and the lower part of the settler is connected with the catalyst first feeding inlet(s) and the regenerator respectively, and the regenerated catalyst outlet of the regenerator is connected with the catalyst second feeding inlet(s).

In the third aspect, the present invention provides a method for preparing low-carbon olefin, which uses the device for preparing low-carbon olefin according to the present invention, the method includes:
reacting the gaseous raw material(s) and the catalyst in the reaction zone of the fluidized bed reactor;
feeding the obtained product and the entrained catalyst to a settler through the top of the reaction zone;
the settler separates the product and the entrained catalyst, and a part of the separated catalyst from the catalyst first feeding inlet(s) is directly supplied into the dense-phase zone formed between the raw material first distributor (8) and the raw material second distributors (11), another part is supplied into the catalyst distribution zone from the catalyst second feeding inlet(s) after being regenerated by the regenerator.

According to the fluidized bed reactor and the device for preparing low-carbon olefin provided by the present invention, by arranging a raw material first distributor, a raw material second distributor and a catalyst distributor in the reaction zone of the fluidized bed reactor, a dense-phase zone and a catalyst distribution zone are formed, so that the unregenerated circulating catalyst supplied from the catalyst first feeding inlet(s) can directly enter the dense-phase zone and contact and react with the raw material(s) of reaction, and the regenerated catalyst supplied from the catalyst second feeding inlet(s) enters into the fluidized bed reactor, then it is pre-distributed in the catalyst distribution zone under the action of the catalyst distributor, so as to realize energy transfer and reaction under the action of the flow field, thereby making the distribution of the regenerated catalyst more uniform, achieving mixing control of the particles of the regenerated catalyst, and improving the reaction efficiency in the fluidized bed reactor.

According to the fluidized bed reactor and the device for preparing low-carbon olefin provided by the present invention, the raw material(s) of reaction are stratified and introduced into the reaction zone through the raw material first distributor and the raw material second distributor, thereby realizing the segmented flow field control of the fluidized bed reactor, which permits to effectively achieve full contact between the catalyst and the raw material(s) of reaction, effectively eliminate the defects of poor fluidization state and low selectivity for low-carbon olefin, further improve the reaction efficiency, and help increase the yield of low-carbon olefin.

Other characters and advantages of the present invention will be described in detail in the following section of embodiments.

The invention thus provides a first series of exemplary embodiments as follows:
1. A fluidized bed reactor, including:
a raw material first distributor (8), a raw material second distributor (11) and a catalyst distributor (16) in the reaction zone of the fluidized bed reactor, which are used to distribute gaseous raw material(s), wherein the raw material first distributor (8) is below the raw material second distributor (11); wherein the raw material first distributor (8) and the raw material second distributor (11) have same or different opening ratio, each independently is 0.05-5%;
a raw material first feeding inlet(s) (1) at the bottom of the fluidized bed reactor, which allows at least a part of the raw material(s) to be distributed twice sequentially through the raw material first distributor (8) and the raw material second distributor (11);
the catalyst distributor (16) includes a catalyst distributor main guide pipe (47), which is coaxially distributed with the raw material first distributor (8) and the raw material second distributor (11), wherein the catalyst distributor main guide pipe (47) passes through the raw material first distributor (8) and the raw material second distributor (11) from bottom to top.
2. The fluidized bed reactor according to the first series of exemplary embodiment 1, characterized in that the fluidized bed reactor has one or more raw material Y-th feeding inlet(s) sequentially arranged from bottom to top above the raw material first feeding inlet(s) (1), wherein Y is a positive integer ≥2, the prerequisite is that when there is one or more raw material Y-th feeding inlets, the arrangement of the raw material first feeding inlet(s) and the raw material Y-th feeding inlet(s) make the ratio of the feed amount of the raw material Y-th feeding inlet(s) to the feed amount of the (Y−1)-th raw material feeding inlet(s) be 1:1-10.
3. The fluidized bed reactor according to the first series of exemplary embodiment 1, characterized in that at least one catalyst first feeding inlet(s) (24) is arranged on the side wall of reactor between the raw material first distributor (8) and the raw material second distributor (11); and
the raw material first distributor (8) includes a first distributor central region (40) and a first distributor external annular region (42) located on the periphery of the first distributor central region (40), the first distributor external annular region (42) is provided with a first distributor enhancement region (38) corresponding to the radial position of the catalyst first feeding inlet(s) (24), and the first distributor enhancement region (38) has a reduced hole diameter relative to other regions of the raw material first distributor (8), so that the catalyst has a substantially uniform distribution in the radial direction between the raw material first distributor (8) and the raw material second distributor (11).
4. The fluidized bed reactor according to the first series of exemplary embodiment 3, characterized in that the first distributor central region (40) is a circle with a radius r, and the first distributor external region (42) is a circular ring with the difference between the outer diameter and the inner diameter being d, wherein r/d=½−⅗, r+d=D, and D is the inner diameter of the fluidized bed reactor; the area of the first distributor enhancement region (38) is set to a ratio of 1/10-½ to the area of the first distributor external region (42).
5. The fluidized bed reactor according to the first series of exemplary embodiment 3, characterized in that the raw material first distributor (8) has region(s) other than the first distributor enhancement region (38), of which the opening ratio is 1.5-10%, preferably 2-5%, and the hole diameter is 2-30 mm, preferably the hole diameter difference between each hole does not exceed ±10%; the opening ratio of the first distributor enhancement region (38) is 0.01-1.5% and the hole diameter is 0.1-20 mm, preferably the hole diameter difference between each hole does not exceed ±10%.
6. The fluidized bed reactor according to the first series of exemplary embodiment 3, characterized in that the first distributor enhancement region (38) is provided with a plurality of columnar first distributor enhanced nozzles (39), the included angle formed by the center line of the first distributor enhanced nozzle(s) (39) and the horizontal direction is 45°-75°.
7. The fluidized bed reactor according to the first series of exemplary embodiment 6, characterized in that the first distributor enhanced nozzle(s) (39) includes an enhanced nozzle inlet (39-1), an enhanced nozzle reducing pipe (39-2), the enhanced nozzle pipe throat (39-3), an enhanced nozzle expansion section (39-4) and an enhanced nozzle outlet (39-5) connected in sequence, wherein the enhanced nozzle inlet (39-1) is connected to the main body of the raw material first distributor (8); wherein,
the included angle range formed by the enhanced nozzle reducing pipe (39-2) and the horizontal direction is 30°-70°, and the included angle range formed by the enhanced nozzle expansion section (39-4) and the horizontal direction is 30°-70°, the ratio of the diameter of the enhanced nozzle pipe throat (39-3) to the diameter of the enhanced nozzle inlet (39-1) is 1:5-20, and the ratio of the length of the enhanced nozzle pipe throat (39-3) to the diameter of the enhanced nozzle pipe throat (39-3) is 5-10:1.
8. The fluidized bed reactor according to any one of the first series of exemplary embodiments 1 to 7, characterized in that the section of the fluidized bed reactor from the raw material first distributor (8) upward to before diameter reduction has a height h, and the raw material second distributor (11) is arranged at an axial distance of ¼-½ h from the raw material first distributor (8).
9. The fluidized bed reactor according to the first series of exemplary embodiment 8, characterized in that the opening ratio of the raw material second distributor (11) is 0.05-5%, preferably 3-5%, and the hole diameter is 1-30 mm, preferably the hole diameter difference between each hole does not exceed ±10%.
10. The fluidized bed reactor according to the first series of exemplary embodiment 8, characterized in that the raw material second distributor (11) is provided with a second distributor gas main guide pipe (43) extending along the radial direction of the raw material second distributor (11), a plurality of the second distributor gas annulus gap guide pipes (44) arranged sequentially along the radial direction of the raw material second distributor (11), and a second distributor tuyere (45) arranged on the second distributor gas annulus gap guide pipe (44) and a second distributor solid guide groove(s) (46), each of the second distributor gas annulus gap guide pipes (44) is arranged to be distributed in an annular shape around the central region of the raw material second distributor (11), and the second distributor solid guide groove (46) is located between the two adjacent the second distributor gas annulus gap guide pipes (44); wherein the fluidized bed reactor has a raw material second feeding inlet (2), which is in fluid connection with the second distributor gas main guide pipe (43).
11. The fluidized bed reactor according to the first series of exemplary embodiment 10, characterized in that the ratio of the width of the second distributor gas annulus gap guide pipe (44) to the width of the second distributor solid guide groove (46) is 1:2-6.
12. The fluidized bed reactor according to any one of the first series of exemplary embodiments 1 to 7, characterized in that the catalyst distributor main guide pipe (47) passes through the raw material first distributor (8) and the raw material second distributor (11) from bottom to top, the section of the fluidized bed reactor from the raw material first distributor (8) upward to before diameter reduction has a height h, the height of the catalyst distributor main guide pipe (47) upward from the raw material first distributor (8) is h1, then ¼h<h1≤¾h.
13. The fluidized bed reactor according to the first series of exemplary embodiment 12, characterized in that the catalyst distributor (16) includes multi-layers of catalyst distribution components (48) distributed along the up and down direction of the catalyst distributor main guide pipe (47), the catalyst distribution components (48) extends in the radial direction, so that the catalyst transported axially along the catalyst distributor main guide pipe (47) can be distributed radially inside the fluidized bed reactor.
15. The fluidized bed reactor according to the first series of exemplary embodiment 13, characterized in that the catalyst distribution components (48) include a plurality of catalyst first distribution conduits (49) and a plurality of catalyst second distribution conduits (50), the catalyst first distribution conduits (49) and the catalyst second distribution conduits (50) are distributed circumferentially and staggered along the catalyst distributor main guide pipe (47) and are both connected to the catalyst distributor main guide pipe (47), and the catalyst first distribution conduits (49) and the catalyst second distribution conduits (50) are respectively provided with a plurality of catalyst outlets (51); wherein the number of the catalyst distribution conduits in each layer of catalyst distribution
components (48) is X (X≥2), the circumferential angle spacing of a plurality of catalyst distribution conduits is in 180°/X distribution; the catalyst outlets (51) have a shape selected from square, circle and polygon.
16. The fluidized bed reactor according to the first series of exemplary embodiment 14, characterized in that the number of the catalyst distribution components (48) is preferably M layers (M≥3), and the catalyst distribution components (48) is the 1st layer, the 2nd layer, . . . the M-th layer from top to bottom; wherein the length of the catalyst distribution conduit in the n-th layer catalyst distribution components (48) is (0.7-0.9)ⁿ*D/2, in which D is the inner diameter of the reactor, and n is the corresponding number of layers.
17. The fluidized bed reactor according to the first series of exemplary embodiment 14, characterized in that the catalyst outlets (51) on the catalyst first distribution conduit(s) (49) and the catalyst second distribution conduit(s) (50) are equidistantly distributed.
18. The fluidized bed reactor according to any one of the first series of exemplary embodiments 3 to 7, characterized in that, a circulating distribution baffle (34) connected to the inner wall of the fluidized bed reactor is provided above the catalyst first feeding inlet(s) (24).
19. The fluidized bed reactor according to the first series of exemplary embodiment 18, characterized in that the ratio of the distance between the circulating distribution baffle (34) and the catalyst first feeding inlet(s) (24) to the hole diameter of the catalyst first feeding inlet(s) (24) is 1-10:1.
20. The fluidized bed reactor according to the first series of exemplary embodiment 18, characterized in that the circulating distribution baffle (34) is provided with a plurality of circulating distribution baffle grooves (37), and the included angle (a) formed by the circulating distribution baffle grooves (37) and the horizontal direction is 30°-75°.
21. A device for preparing low-carbon olefin, characterized in that the device includes a fluidized bed reactor (7) according to any one of the first series of exemplary embodiments 1 to 19, a settler (9), and a regenerator (10), at least one catalyst first feeding inlet(s) (24) for the first feeding of catalyst into the region between the raw material first distributor (8) and the raw material second distributor (11) is provided on the side wall of the reactor, and a catalyst second feeding inlet(s) (27) for the second feeding of catalyst into the distributor main guide pipe (47) is provided at the bottom of the reactor; wherein:
the settler (9) is connected to the upper part of the reaction zone of the fluidized bed reactor (7), and the lower part of the settler (9) is connected to the catalyst first feeding inlet(s) (24) and the regenerator (10) respectively, and the regenerated catalyst outlet of the regenerator (10) is connected to the catalyst second feeding inlet(s) (27).
22. The device according to the first series of exemplary embodiment 21, characterized in that the number of the catalyst first feeding inlet(s) (24) is k, k≥2, and preferably k≤12; the included angle formed by the center lines of each of the catalyst first feeding inlet(s) (24) is 360°/k.
23. The device according to the first series of exemplary embodiment 22, characterized in that a lower section of settler (17), an upper section of settler (18) located above the lower section of settler (17) and the settler cyclone separator (19) located in the upper section of settler (18) are provided in the settler (9), and the gas outlet of the settler cyclone separator (19) is connected to the product gas outlet (5) of the settler (9), a settler distribution plate (12) is provided at the lower part of the lower section of settler (17), and the lower part of the settler distribution plate (12) is connected to the catalyst first feeding inlet(s) (24) through a circulation pipe(s) (22), and is connected to the regenerator (10) through the stripper (21).
24. The device according to the first series of exemplary embodiment 23, characterized in that the settler distribution plate (12) is provided with a settler first distribution plate holes (35) and a settler second distribution plate holes (36), the settler first distribution plate holes (35) and the settler second distribution plate holes (36) are respectively arranged to be annularly distributed around the central region of the settler distribution plate (12), the size ratio of the settler first distribution plate holes (35) to the settler second distribution plate holes(36) is 1-3:4.
25. The device according to any one of the first series of exemplary embodiments 21 to 24, characterized in that the top of the fluidized bed reactor (7) is provided with a separation riser (15) extending into the settler (9), and a riser baffle (14) located above the outlet of the separation riser (15) is provided in the settler (9).
26. A method for preparing low-carbon olefin in the device of any one of the first series of exemplary embodiments 21-24, comprising:
the gaseous raw material(s) and the catalyst are reacted in the reaction zone of the fluidized bed reactor;
the obtained product and the entrained catalyst are fed into a settler through the top of the reaction zone;
the product and the entrained catalyst are separated in the settler, wherein a part of the separated catalyst is directly supplied into the dense-phase zone formed between the raw material first distributor (8) and the raw material second distributor (11) through the catalyst first feeding inlet, and another part is fed into the catalyst distribution zone through the catalyst second feeding inlet(s) after being regenerated by the regenerator.
27. The method according to the first series of exemplary embodiment 26, characterized in that the linear velocity of the material(s) in the dense-phase zone is 1-10 m/s.
28. The method according to the first series of exemplary embodiment 26, characterized in that, in the catalyst obtained by the separation, the mass ratio of the part fed into the dense-phase zone to the part fed into the regenerator is 1:0.2-1.
29. The method according to the first series of exemplary embodiment 26, characterized in that, the ratio of the pressure drop produced by the gaseous raw material(s) when passing through the dense-phase zone to the pressure drop produced by the gaseous raw material(s) when passing through the catalyst distribution zone is 1.5-4:1.
30. The method according to the first series of exemplary embodiment 26, characterized in that, after the gaseous raw material(s) pass through the raw material first distributor, the included angle formed by the annulus gap space velocity and the horizontal direction is 45°-75°, the ratio of the internal and external porosity fluctuations is 0.9-0.95:1.

### DESCRIPTION OF FIGURES

The figures are used to provide a further understanding of the present invention and constitute a part of the specification. They are used to explain the present invention together with the following specific embodiments, but do not constitute a limitation of the present invention. In the figures:

FIG. 1 is a schematic diagram of a device for preparing low-carbon olefin according to a specific embodiment of the present invention;

FIG. 2 is a structural schematic diagram of a specific embodiment of the raw material first distributor in the device for preparing low-carbon olefin according to the present invention;

FIG. 3 is a structural schematic diagram of another specific embodiment of the raw material first distributor in the device for preparing low-carbon olefin according to the present invention;

FIG. 4 is a structural schematic diagram of a specific embodiment of the first distributor enhanced nozzle in the device for preparing low-carbon olefin according to the present invention;

FIG. 5 is a structural schematic diagram of a specific embodiment of the raw material second distributor in the device for preparing low-carbon olefin according to the present invention;

FIG. 6 is a structural schematic diagram of a specific embodiment of the catalyst distributor in the device for preparing low-carbon olefin according to the present invention;

FIG. 7 is a structural schematic diagram of another specific embodiment of the catalyst distributor in the device for preparing low-carbon olefin according to the present invention;

FIG. 8 is a distribution diagram in the fluidized bed reactor when the circulating distribution baffles are set to two in the device for preparing low-carbon olefin according to the present invention;

FIG. 9 is a distribution diagram in the fluidized bed reactor when the circulating distribution baffles are set to four in the device for preparing low-carbon olefin according to the present invention;

FIG. 10 is a structural schematic diagram of a specific embodiment of the circulating distribution baffle in the device for preparing low-carbon olefin according to the present invention;

FIG. 11 is a structural schematic diagram illustrating a catalyst distributor according to the present invention;

FIG. 12 is a structural schematic diagram of a specific embodiment of the settler distribution plate in the device for preparing low-carbon olefin according to the present invention;

FIG. 13 is a schematic diagram showing how the unevenness of regenerated catalyst distribution changes with height according to Embodiment 1 of the present invention;

FIG. 14 is a comparative chart of the unevenness variation of regenerated catalyst distribution with height in Example 1 of the present invention and Comparative Example 3.

Explanations of reference signs:

| | |
|---|---|
| 1. Raw material first feeding inlet | 2. Raw material second feeding inlet |
| 3. Stripping medium | 4. Regenerator gas |
| 5. Product gas outlet | 6. Flue gas outlet |
| 7. Fluidized bed reactor | 8. Raw material first distributor |
| 9. Settler | 10. Regenerator |
| 11. Raw material second distributor | 12. Settler distribution plate |
| 13. Regenerator gas distributor | 14. Riser baffle |
| 15. Separation riser | 16. Catalyst distributor |
| 17. Lower section of settler | 18. Upper section of settler |
| 19. Settler cyclone separator | 20. Regenerator cyclone separator |
| 21. Stripper | 22. Circulation pipe |
| 23. Circulation pipe control valve | 24. Catalyst first feeding inlet(s) |
| 25. Regenerator circulation discharge pipe | 26. Regenerator circulation discharge pipe |
| control valve | |
| 27. Catalyst second feeding inlet(s) | 28. Dense-phase zone |
| 29. Catalyst distribution zone | 30. Stripper feed pipe |
| 31. Stripper discharge pipe | 32. Stripper control valve |
| 33. Regenerator feed pipe | 34. Circulating distribution baffle |
| 35. Settler first distribution plate hole | 36. Settler second distribution plate pore |
| 37. Circulating distribution baffle groove | 38. First distributor enhancement region |
| 39. First distributor enhanced nozzle | 39-1. Enhanced nozzle inlet |
| 39-2. Enhanced nozzle reducing pipe | 39-3. Enhanced nozzle pipe throat |
| 39-4. Enhanced nozzle expansion section | 39-5. Enhanced nozzle outlet |
| 40. First distributor central region | 41. First distributor central region tuyere |
| 42. First distributor external annular region | 43. Second distributor gas main guide pipe |
| 44. Second distributor gas annulus gap guide pipe | 45. Second distributor tuyere |
| 46. Second distributor solid guide groove | 47. Catalyst distributor main guide pipe |
| 48. Catalyst distribution component | 49. catalyst first distribution conduit |
| 50. catalyst second distribution conduit | 51. Catalyst outlet |
| α. Angle formed by the circulating | R. Radius of the circulating distribution |
| distribution baffle groove and the | baffle |
| horizontal direction | |

### DETAILED DESCRIPTION OF THE INVENTION

Specific embodiments of the present invention will be described in detail below. It should be understood that the specific embodiments described here are only used to illustrate and explain the present invention, and are not intended to limit the present invention.

In the present specification, unless otherwise stated, all technical features and preferred features mentioned in the present specification regarding various aspects, various series and/or various embodiments can be combined with each other to form new technical solutions.

In the present specification, unless otherwise stated, the specific steps, specific values, and specific materials described in the examples can be combined with other features in other parts of the specification. For example, if the description of invention or the section of specific embodiments mentions that the reaction temperature is 10-100° C., and the specific reaction temperature recorded in the examples is 20° C., then it can be considered that the present specification has specifically disclosed the range of 10-20° C., or the range of 20° C.-100° C., and this range can be combined with other features in other parts of the specification to form new technical solutions.

The endpoints of ranges and any values disclosed herein are not limited to the precise ranges or values, but these ranges or values are to be understood to include values approaching such ranges or values. For numerical ranges, the endpoint values of each range, the endpoint values of each range and individual point values, and the individual point values can be combined with each other to obtain one or more new numerical ranges, and these numerical ranges shall be deemed to be specifically disclosed herein.

In the present invention, unless otherwise specified, the directional words used such as “upper” and “lower” usually refer to upper and lower as shown in the figures; “inner” and “outer” refer to inside and outside relative to the silhouette of each component itself.

In the present specification, unless otherwise stated, the terms “include(s)”, “comprise(s)”, “contain(s)”, “have(has)” and similar expressions indicate open-ended situations, but should also be understood to also expressly disclose closed-ended situations. For example, “include” indicates that other elements not listed may also be included, but it also explicitly discloses that only the listed elements are included.

As used herein, the “gaseous raw material(s)” has a meaning well known in the art. In particular, the gaseous raw material(s) used in the fluidized bed reactor of the present invention may optionally contain a certain amount of liquid, especially in the form of dispersed droplets, as long as the presence of the liquid does not substantially hinder the fluidized state of the fluidized bed.

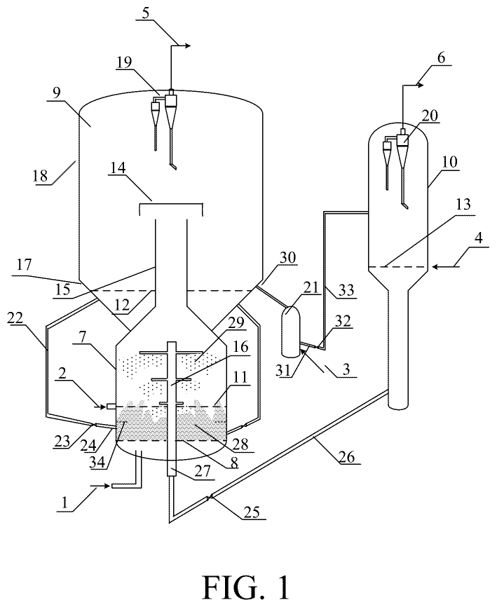

As shown in FIG. 1, the reaction zone of the fluidized bed reactor 7 according to the first aspect of the present invention is provided with a raw material first distributor 8, a raw material second distributor 11 and a catalyst distributor 16 from bottom to top, wherein the catalyst distributor 16 is connected to the catalyst second feeding inlet(s) 27; a dense-phase zone 28 is formed between the raw material first distributor 8 and the raw material second distributor 11; the area where the catalyst distributor 16 is located forms the catalyst distribution zone 29 connected to the dense-phase zone 28; and at least one catalyst first feeding inlet(s) 24 is provided on the side wall of reactor in the dense-phase zone 28.

According to the present invention, by arranging the raw material first distributor 8, the raw material second distributor 11 and the catalyst distributor 16 in the reaction zone of the fluidized bed reactor 7, a dense-phase zone 28 and a catalyst distribution zone 29 are formed in the reaction zone, so that the catalyst supplied from the catalyst first feeding inlet(s) 24 can directly enter the dense-phase zone 28 and perform a contact and reaction with the raw material(s) of reaction; the catalyst supplied from the catalyst second feeding inlet(s) 27 enters the fluidized bed reactor 7 and then passes through the catalyst distributor 16 to perform pre-distribution in the catalyst distribution zone 29, to achieve energy transfer and reaction under the action of the flow field, thereby making the catalyst distribution more uniform, achieving particle mixing control of the catalyst, and improving the reaction efficiency of the fluidized bed reactor 7; the raw material(s) of reaction are stratified into the reaction zone through the raw material first distributor 8 and the raw material second distributor 11, achieving segmented flow field control of the fluidized bed reactor 7, which can effectively realize the full contact of the catalyst and the raw material(s) of reaction, further improve the reaction efficiency and be conducive to increasing the yield of low-carbon olefin.

In the present invention, the catalyst first feeding inlet(s) 24 and the catalyst second feeding inlet(s) 27 of the fluidized bed reactor 7 can be used to supply the same catalyst or different catalysts; when the fluidized bed reactor 7 is applied in a device for preparing low-carbon olefin, the catalyst first feeding inlet(s) 24 can be used to feed unregenerated circulating catalyst, and the catalyst second feeding inlet(s) 27 can be used to feed regenerated catalyst.

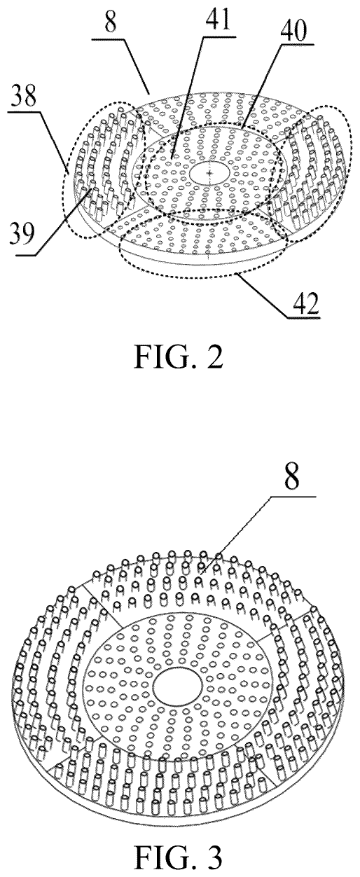

As a specific embodiment of the raw material first distributor 8 in the fluidized bed reactor according to the present invention (referring to FIGS. 2 to 4), the raw material first distributor 8 includes a first distributor central region 40 and a first distributor external annular region 42 located at the outer periphery of the first distributor central region 40, the first distributor external annular region 42 is provided with a first distributor enhancement region 38 corresponding to the radial position of the catalyst first feeding inlet(s) 24.

Referring to FIGS. 2 and 3, the first distributor enhancement region 38 has a reduced hole diameter relative to other regions of the raw material first distributor 8, so that the catalyst has a substantially uniform distribution in the radial direction between the raw material first distributor 8 and the raw material second distributor 11. Preferably, the first distributor enhancement region 38 is arranged in one-to-one correspondence with the catalyst first feeding inlet(s) 24, so that the first distributor enhancement region 38 can quickly mix the raw material(s) of reaction it receives with the catalyst supplied from the catalyst first feeding inlet(s) 24.

In a preferable case, the first catalyst feed port 24 is located above the center of the outer edge of its corresponding first distributor enhancement region 38 to further improve the uniformly mixing effect of the raw material(s) of reaction with the catalyst supplied from the catalyst first feeding inlet(s) 24 by the first distributor enhancement region 38. More preferably, the area of the first distributor enhancement region 38 may be set to a ratio of 1/10 to ½ to the area of the first distributor external annular region 42.

In one embodiment, the first distributor central region 40 is a circle with a radius r, and the first distributor external region 42 is a circular ring with a difference d between the outer diameter and the inner diameter, where r/d=½-⅗, r+d=D, wherein D is the inner diameter of the fluidized bed reactor; the ratio of the area of the first distributor enhancement region 38 to the area of the first distributor external region 42 is 1/N, and N is a natural number of 2, 3 . . . .

In one embodiment, the opening ratio of the region of the raw material first distributor 8 except the first distributor enhancement region (38) is 1.5-10%, preferably 2-5%, and the hole diameter is 2-30 mm, preferably the difference between the hole diameter of each hole does not exceed ±10%; the opening ratio of the first distributor enhancement region 38 is 0.05-1.5%, and the hole diameter is 0.1-20 mm, preferably the difference between the hole diameter of each hole does not exceed ±10%.

According to the present invention, the first distributor central region tuyere 41 on the first distributor central region 40 can be circular, triangular, square, hexagonal, etc., and it has an effective diameter of 0.1-10 mm and an opening ratio of 0.05-5%.

Further preferably, the first distributor enhancement region 38 is provided with a plurality of columnar first distributor enhanced nozzles 39, and the included angle formed by the center line of the first distributor enhanced nozzle 39 and the horizontal direction is 45°-75°, the first distributor enhanced nozzle 39 can form a stronger mixing force between the raw material(s) of reaction distributed by it and the catalyst supplied from the catalyst first feeding inlet(s) 24. The effective diameter of the first distributor enhanced nozzle 39 can be set to 0.1-10 mm, and the opening ratio is 0.05-5%; the distribution holes in the area of the first distributor external annular region 42 that does not correspond to the catalyst first feeding inlet(s) 24 (i.e. the area that is not set as the first distributor enhancement region 38) has same size and opening ratio as those of the first distributor central region tuyere 41.

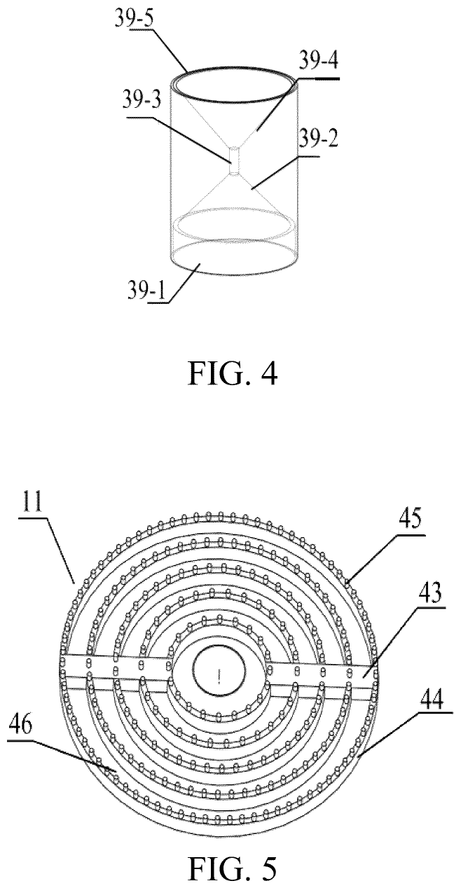

As a preferred embodiment of the first distributor enhanced nozzle 39 in the present invention, referring to FIG. 4, the first distributor enhanced nozzle 39 includes an enhanced nozzle inlet 39-1, an enhanced nozzle reducing pipe 39-2, an enhanced nozzle pipe throat 39-3, an enhanced nozzle expansion section 39-4 and the enhanced nozzle outlet 39-5 that are connected in sequence, and the enhanced nozzle inlet 39-1 are connected to the main body of the raw material first distributor 8. The raw material(s) entering the first distributor 8 enters through the enhanced nozzle inlet 39-1, and is transmitted through the enhanced nozzle reducing pipe 39-2, the enhanced nozzle pipe throat 39-3, and the enhanced nozzle expansion section 39-4 in sequence, and then is sprayed out through the enhanced nozzle outlet 39-5.

Specifically, the included angle range formed by the enhanced nozzle reducing pipe 39-2 and the horizontal direction is between 30° and 70°, and the included angle formed by the enhanced nozzle expansion section 39-4 and the horizontal direction is between 30° and 70°, the ratio of the diameter of the enhanced nozzle pipe throat 39-3 to the diameter of the enhanced nozzle inlet 39-1 is 1:5-20, and the ratio of the length of the enhanced nozzle pipe throat 39-3 to the diameter of the enhanced nozzle pipe throat 39-3 is 5-10:1. The “horizontal direction” in the present invention specifically refers to the direction in which the horizontal plane extends when the fluidized bed reactor 7 is placed on a horizontal plane.

In one embodiment, the section of the fluidized bed reactor from the raw material first distributor 8 upward to before diameter reduction has a height h, and the raw material second distributor 11 is arranged at an axial distance of ¼-½h from the raw material first distributor 8.

As a specific embodiment of the raw material second distributor 11 in the fluidized bed reactor of the present invention, the opening ratio of the raw material second distributor 11 is 0.05-5%, preferably 3-5%, and the hole diameter is 1-30 mm, and preferably the hole diameter difference between each hole does not exceed #10%. Referring to FIG. 5, the raw material second distributor 11 is provided with a second distributor gas main guide pipe 43 extending in the radial direction of the raw material second distributor 11, and a plurality of second distributor gas annulus gap guide pipes 44 arranged in sequence along the radial direction of the raw material second distributor 11, the second distributor tuyere 45 provided on the second distributor gas annulus gap guide pipe 44 and the second distributor solid guide groove 46, each second distributor gas annulus gap guide pipes 44 are arranged in an annular distribution around the central region of the raw material second distributor 11, and the second distributor solid guide groove 46 is located between the two adjacent second distributor gas annulus gap guide pipes 44; wherein the fluidized bed reactor has a raw material second feeding inlet 2, which is in fluid connection with the second distributor gas main guide pipe 43. The opening of the second distributor tuyere 45 is horizontal, and it can be circular, triangular, square, hexagonal, etc., the effective diameter is 0.1-10 mm, and the opening ratio is 0.05-5%.

Preferably, the ratio of the width of the second distributor gas annulus gap guide pipe 44 to the width of the second distributor solid guide groove 46 is 1:2-6.

In the present invention, the catalyst distributor 16 includes a catalyst distributor main guide pipe 47. The catalyst distributor 16 may have different structures as long as the catalyst transported axially along the catalyst distributor main guide pipe 47 can be distributed radially inside the fluidized bed reactor.

As a specific embodiment of the catalyst distributor 16 in the fluidized bed reactor of the present invention, the catalyst distributor main guide pipe 47 passes through the raw material first distributor 8 and the raw material second distributor 11 from bottom to top, the section of the fluidized bed reactor from the raw material first distributor 8 upward to before diameter reduction has a height h, and the height of the catalyst distributor main guide pipe 47 upward from the raw material first distributor 8 is h1, then ¼h<h1≤¾h.

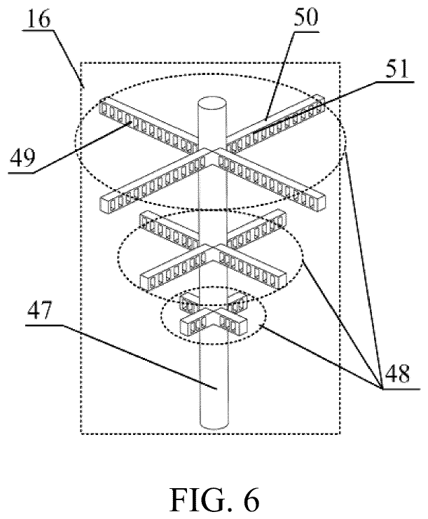

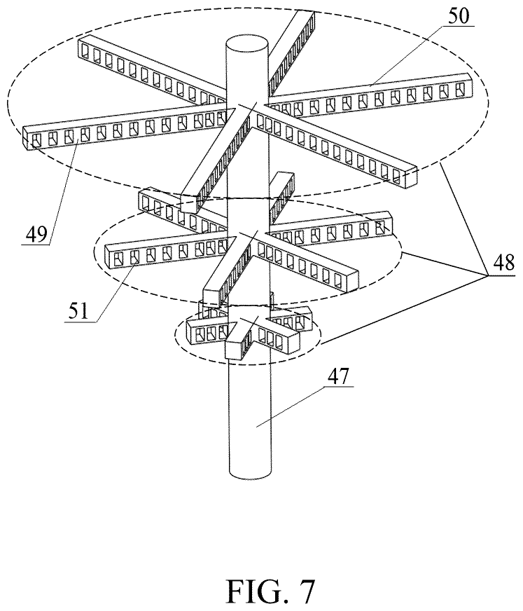

In one embodiment, referring to FIGS. 6 and 7, the catalyst distributor 16 is a dendritic arrangement scheme, which includes a catalyst distributor main guide pipe 47 and multiple layers of catalyst distribution components 48 distributed along the up and down direction of the catalyst distributor main guide pipe 47; the catalyst distributor main guide pipe 47 are arranged vertically in the reaction zone and connected with the catalyst second feeding inlet(s) 27; the catalyst distribution components 48 extends radially to enable radial distribution of the catalyst transported axially along the catalyst distributor main guide pipe 47 within the fluidized bed reactor.

In one embodiment, the catalyst distribution components (48) include a plurality of catalyst first distribution conduits 49 respectively extending radially outward from the distributor main conduit 47 and a plurality of catalyst second distribution conduits 50. The catalyst first distribution conduit(s) 49 and the catalyst second distribution conduit(s) 50 are distributed circumferentially and staggered along the catalyst distributor main conduit 47 and are both connected to the catalyst distributor main conduit 47; the catalyst first distribution conduit(s) 49 and the catalyst second distribution conduit(s) 50 are respectively provided with multiple catalyst outlet 51. The catalyst enters each catalyst distribution components 48 from the catalyst distributor main guide pipe 47, is transported through the catalyst first distribution conduit(s) 49 and the catalyst second distribution conduit(s) 50, and enters the reactor through the catalyst outlet 51.

In one embodiment, the number of catalyst distribution conduits in each layer of catalyst distribution component 48 is X (X≥2 and ≤15), the circumferential angle spacing of multiple catalyst distribution conduits is in 360°/X distribution; the catalyst outlet 51 has a shape selected from square, circle and polygon.

Preferably, the number of the catalyst distribution components 48 is preferably M layers (M≥3 and ≤10), and the catalyst distribution components 48 are the 1st layer, the 2nd layer . . . the M-th layer from top to bottom; wherein the length of the catalyst distribution conduit in the n-th layer catalyst distribution components 48 is (0.7-0.9)ⁿ*D/2, D is the inner diameter of the reactor, and n is the corresponding number of layers, for example, the diameter of the distribution conduit is 0.75ⁿ*D/2.

In preferable case, the lengths of the catalyst first distribution conduit(s) 49 and the catalyst second distribution conduit(s) 50 corresponding to the catalyst distribution components 48 decrease from top to bottom, and the catalyst outlets 51 on the catalyst first distribution conduit(s) 49 and the catalyst second distribution conduit(s) 50 are in equally spaced distribution.

By way of example, the catalyst first distribution conduit(s) 49 and the catalyst second distribution conduit(s) 50 can be collectively referred to as catalyst distribution conduits. The number of catalyst distribution components 48 is preferably M layers (M≥3 and ≤10), and the reactor inner diameter is D meters. The catalyst distribution components 48 is the first layer, the second layer, the third layer . . . the M-th layer from top to bottom, and the diameter of the catalyst distribution conduit in each layer of the catalyst distribution components 48 is 0.75″ *D meters (n is corresponding number of layers); the number of catalyst distribution conduits in each layer of catalyst distribution component 48 is X (X≥2 and ≤15), and the circumferential angle spacing of the plurality of catalyst distribution conduits is in 360°/X distribution; and the catalyst outlet 51 can be square, circular, or polygon etc.; the diameter of the effective channel is 20-100 mm; the ratio of the center distance between two adjacent catalyst outlets 51 to the width of the catalyst outlet 51 along its distribution direction is 1.5-5:1.

In one embodiment, referring to FIG. 11A, catalyst distributor 16 is a main guide pipe arrangement scheme, which includes or consists solely of catalyst distributor main guide pipe 47. In this case, the catalyst distributor main guide pipe 47 is generally tubular in shape, with an upper top open, and the catalyst is transported from bottom to top along the catalyst distributor main guide pipe 47, and enters the fluidized bed reactor through the opening at the top. In an exemplary embodiment, optionally, there are opening(s) on the wall of the catalyst distributor main guide pipe 47, for example, the opening ratio is 5-30%, so that the catalyst can be evenly distributed in the radial direction through the openings.

In one embodiment, referring to FIG. 11B, the catalyst distributor 16 is an inner baffle arrangement scheme, which includes a catalyst distributor main guide pipe 47 and a catalyst main guide pipe inner baffle 47-2. The shape of the catalyst main guide pipe inner baffle 47-2 is not specifically limited, for example, it can be a substantially circular shape, an ellipse (for example, when it is in the installation position, the projection on the horizontal plane is a circle), etc., and its projected area on the horizontal plane is 1/10-¼ of the inner cross section of the catalyst distributor main guide pipe 47. The catalyst main guide pipe inner baffle 47-2 extends obliquely downward from the contact point with the pipe wall of the catalyst distributor main guide pipe 47, and the angle with the vertical direction of the pipe wall is 10-75°, for example, 30-45°.

In one embodiment, referring to FIG. 11C, the catalyst distributor 16 is a secondary distribution arrangement scheme, which includes a catalyst distributor main guide pipe 47 and 2 or more, such as 2-12, such as 3-6 dispersed secondary distribution guide pipes 47-3 connected to the top of the catalyst distributor main guide pipe 47. The outer wall of each secondary distribution flow pipe 47-3 is connected to the outer wall of the catalyst distributor main guide pipe 47 in an arc shape of approximately ¼ of a circle, the radius of the circle is the distance from the center of the horizontal ring at the outlet of the secondary distribution flow pipe to the centerline of the catalyst distributor main guide pipe. The portion of each secondary distribution flow pipe 47-3 other than the arc connecting portion is a straight circular tube connected with an arc. A plurality of secondary distribution guide pipes 47-3 are arranged at even intervals on the circumference. Preferably, the plurality of secondary distribution guide pipes 47-3 have consistent shapes and sizes. Preferably, the length of the secondary distribution flow pipe 47-3 can be specifically determined according to the actual situation, for example, the length thereof extending in the radial direction is (0.1-0.9)*D/2, wherein D is the inner diameter of the reactor.

In one embodiment, referring to FIG. 11D, the catalyst distributor 16 is a spiral guide pipe arrangement scheme, which includes a catalyst distributor main guide pipe 47 and one layer or multi-layers of spiral guide pipe 47-4 distributed axially along the catalyst distributor main guide pipe 47. Each layer contains 2 or more, such as 2-15, such as 3 or 4 dispersed spiral guide pipes 47-4. The plurality of spiral guide pipes 47-4 in each layer are evenly spaced on the circumference. Preferably, the catalyst distributor 16 includes 2-10 layers of spiral guide pipes 47-4. Preferably, the length of each layer of secondary distribution guide pipes 47-4 can be specifically determined according to the actual situation, for example, the length extending in the radial direction is (0.5-0.9)ⁿ*D/2, D is the inner diameter of the reactor, n is the corresponding number of layers.

In one embodiment, referring to FIG. 11E, the catalyst distributor 16 is an annular guide pipe arrangement scheme, which includes a catalyst distributor main guide pipe 47 and one layer or multi-layer annular guide pipe 47-5 axially distributed along the catalyst distributor main guide pipe 47. The annular guide pipe includes a guide ring 47-6 and a connecting pipe 47-7 connecting it with the catalyst distributor main guide pipe 47. The connecting pipe 47-7 is preferably in a spiral structure. Each layer of annular guide pipes includes 2 or more, for example 2-10, such as 2-6, such as 3 or 4 dispersed connecting pipes 47-7. The connecting pipes 47-7 and 47-4 of each layer are arranged at even intervals on the circumference. Preferably, the catalyst distributor 16 includes 2-10 layers, such as 2-6 layers of annular guide pipes 47-5. Preferably, the diameter of each layer of annular guide pipe 47-5 is (0.5-0.9)ⁿ*D/2, wherein D is the inner diameter of the reactor, and n is the corresponding number of layers.

In each arrangement scheme of the catalyst distributor 16 above, referring to the illustration in the dendritic arrangement scheme, “n” indicating the number of layers counts the corresponding components from top to bottom.

The fluidized bed reactor has one or more raw material Y-th feeding inlet(s) arranged sequentially from bottom to top above the raw material first feeding inlet(s) 1, wherein Y is a positive integer ≥2, provided that when there are one or more raw material Y-th inlet(s), the arrangement of the raw material first feeding inlet(s) and the raw material Y-th feeding inlet(s) is such that the ratio of the feeding amount of the raw material Y-th feeding inlet(s) to the feeding amount of the raw material (Y−1)-th feeding inlet(s) is 1:1-10. Preferably, Y≤10, preferably ≤5, for example, the fluidized bed reactor has a total of 2 or 3 raw material feeding inlets.

According to the present invention, the fluidized bed reactor 7 is provided with corresponding feeding inlet(s) to provide reaction raw materials to the raw material first distributor 8 and the raw material second distributor 11. As a preferred embodiment of the feeding inlet(s) in the present invention, the fluidized bed reactor 7 is provided with a raw material first feeding inlet(s) 1 and a raw material second feeding inlet(s) 2. The raw material first feeding inlet(s) 1 is connected to the bottom of the raw material first distributor 8, and the raw material second feeding inlet(s) 2 is located in the intersection area of the dense-phase zone 28 and the catalyst distribution zone 29. The feeding inlets in the present invention are not limited to only providing the raw material first feeding inlet(s) 1 and the raw material second feeding inlet(s) 2, and other feeding inlets can be provided according to the input requirements of the raw material(s) of reaction.

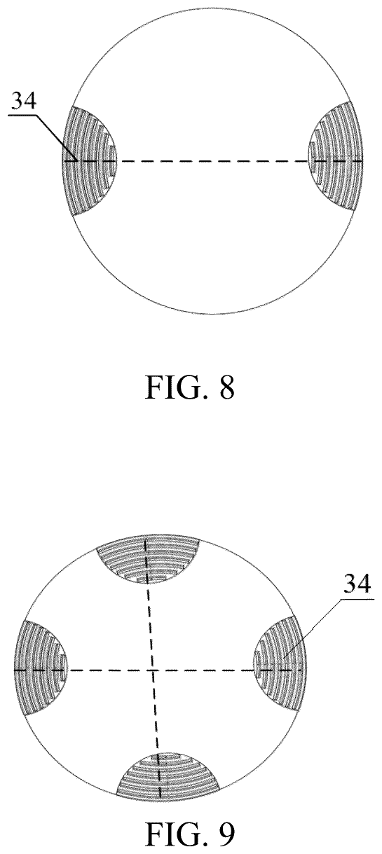

As a preferred embodiment of the fluidized bed reactor 7 in the present invention, referring to FIGS. 8 to 10, a circulating distribution baffle 34 connected to the inner wall of the fluidized bed reactor 7 is provided above the catalyst first feeding inlet(s) 24. The circulating distribution baffle 34 is arranged in one-to-one correspondence with the catalyst first feeding inlet(s) 24 and is located directly above the corresponding catalyst first feeding inlet(s) 24.

In preferable case, the ratio of the distance between the circulating distribution baffle 34 and the catalyst first feeding inlet(s) 24 to the hole diameter of the catalyst first feeding inlet(s) 24 is 1-10:1. The circulating distribution baffle 34 is specifically a structure formed by intersecting a circle whose center is located on the inner wall of the fluidized bed reactor 7 and whose radius is R with the inner wall of the fluidized bed reactor 7, wherein the ratio of the size of R to the radius size of the reactor is 1:4-10.

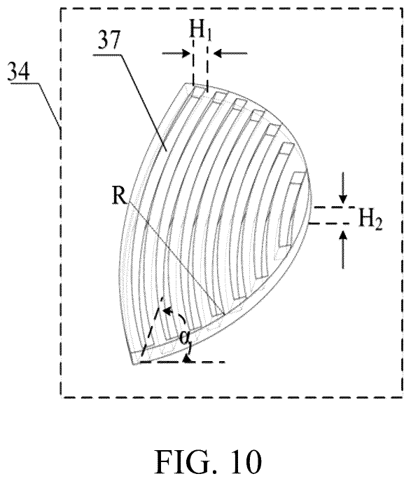

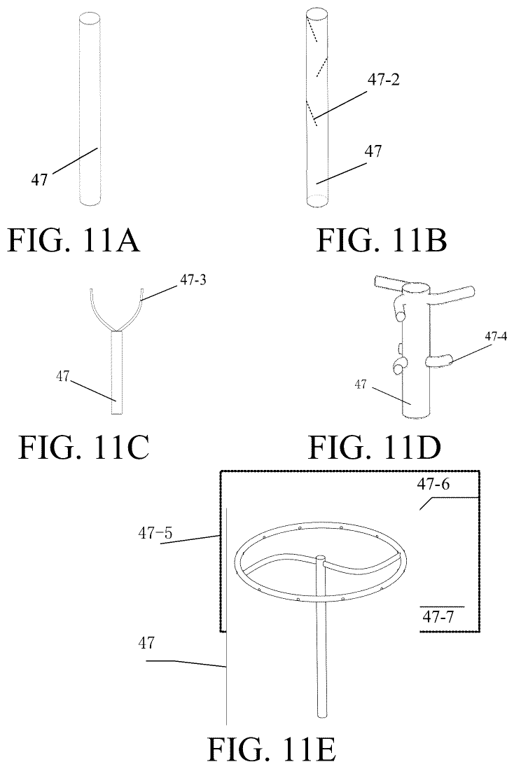

Referring to FIG. 10, the circulating distribution baffle 34 is provided with a plurality of circulating distribution baffle grooves 37, wherein the angle α formed between the circulating distribution baffle grooves 37 and the horizontal direction is 30°-75°, so that the circulating distribution baffle grooves 37 faces the center of the fluidized bed reactor 7. The circulating distribution baffle 34 can distribute the catalyst supplied from the catalyst first feeding inlet(s) 24 to the center of the fluidized bed reactor 7, the flow direction of the catalyst is strengthened by the circulating distribution baffle groove 37, so that the uniformity of catalyst distribution is further improved. The width of the circulating distribution baffle groove 37 is H₁, and the ratio of the size of H₁ to the radius R of the circulating distribution baffle 34 is 0.01-0.1:1; the height of the circulating distribution baffle 34 is H₂, and the ratio of the size of H₂ to the radius R of circulating distribution baffle 34 is 0.001-0.05:1.

In the second aspect, the present invention provides a device for preparing low-carbon olefin; referring to FIG. 1, the device includes a fluidized bed reactor 7 provided by any of the above technical solutions, a settler 9 and a regenerator 10. The side wall of the reactor is provided with at least one catalyst first feeding inlet(s) 24 for feeding the first catalyst to the location between the raw material first distributor 8 and the raw material second distributor 11, and the bottom of the reactor is provided with a second catalyst feed pipe 27 for feeding the second catalyst feed to the distributor main guide pipe 47; wherein the settler 9 is connected to the upper part of the reaction zone of the fluidized bed reactor 7, and the lower parts of the settler 9 are respectively connected with the catalyst first feeding inlet(s) 24 and the regenerator 10, and the regenerated catalyst outlet of the regenerator 10 is connected with the catalyst second feeding inlet(s) 27.

In the device for preparing low-carbon olefin of the present invention, the number of the catalyst first feeding inlet(s) 24 is k, k≥2, and preferably k≤12; the angle formed by the center lines of each catalyst first feeding inlet(s) (24) is 360°/k. The lower part of the settler 9 is connected to the catalyst first feeding inlet(s) 24 through circulation pipe(s) 22. Correspondingly, the number of circulation pipe(s) 22 is the same as the number of the catalyst first feeding inlet(s) 24. The circulation pipe(s) 22 is provided with a circulation pipe control valve(s) 23 to be able to control the supply amount from the catalyst first feeding inlet(s) 24 to the dense-phase zone 28.

In the device for preparing low-carbon olefin according to the present invention, the settler 9 can adopt a settling device with a conventional structure to separate the product coming from the fluidized bed reactor 7 and the entrained catalyst. In preferable case, the settler 9 is provided with a lower section of settler 17, an upper section of settler 18 above the lower section of settler 17, and a settler cyclone separator 19 located at the upper section of settler 18. The gas outlet of the settler cyclone separator 19 is connected to the product gas outlet 5 of the settler 9. A settler distribution plate 12 is provided at the lower part of the lower section of settler 17, the top of the settler distribution plate 12 is connected to the circulation pipe(s) 22 and is connected to the regenerator 10 through the stripper 21.

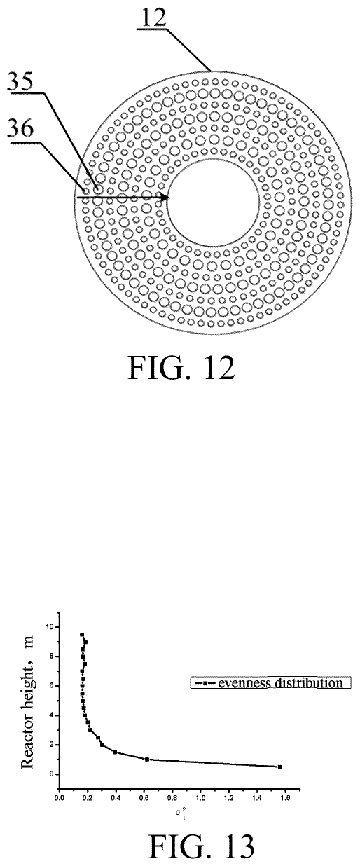

In preferable case, the top of the upper section of settler 18 is hemispherical; referring to FIG. 12, the settler distribution plate 12 is provided with a settler first distribution plate holes 35 and a settler second distribution plate holes 36. The settler first distribution plate holes 35 and the settler second distribution plate holes 36 are respectively arranged to be annularly distributed around the central region of the settler distribution plate 12, and the size ratio of the settler first distribution plate holes 35 to the settler second distribution plate holes 36 is 1-3:4. The settler first distribution plate holes 35 and the settler second distribution plate holes 36 are preferably distributed alternately in an annular shape on the settler distribution plate 12, and the opening ratio of the two is 0.05-5%. The upper section of settler 18 adopts a hemispherical design. Compared with the structure of the traditional settler, the size of the settler 9 can be reduced by 10-21% under the same volume. The dome-shaped design of the upper section 18 of the settler can make the gas flow field in the settler 9 more stable.

According to the present invention, the settler cyclone separator 19 may adopt a conventional cyclone separation device to effectively separate the product from the entrained catalyst. Exemplarily, the settler cyclone separator 19 is configured as a two-stage or multi-stage cyclone separator in series, wherein the inlet of the first-stage cyclone separator is connected to the region of the upper section of settler 18, the gas outlet in the first-stage cyclone separator is connected to the inlet of the adjacent cyclone separator, and the product gas is obtained from the gas outlet of the next-stage cyclone separator; the solid outlets of all cyclone separators are connected to the settler 9 region; the gas outlet of the last stage cyclone separator is connected with the product gas outlet 5 of the settler 9, so that the product gas obtained by the last stage cyclone separator is discharged through the product gas outlet 5.

Referring to FIG. 1, the lower part of the settler 9 is connected to the regenerator 10 through the stripper 21; the specific connection mode can be: the lower part of the settler 9 is connected to the stripper 21 through the stripper feed pipe 30; the stripper 21 is provided with a stripping medium inlet to introduce the stripping medium 3 into the stripper 21; the discharge outlet of the stripper 21 is connected to the regenerator feed pipe 33 of the regenerator 10 through the stripper discharge pipe 31; a stripper control valve 32 is provided on the stripper discharge pipe 31 to control the amount of feed entering the regenerator 10 from the regenerator feed pipe 33.

According to the present invention, the regenerator 10 is provided with a regenerator gas distributor 13 and a regenerator cyclone separator 20 located above the regenerator gas distributor 13; the gas outlet of the regenerator cyclone separator 20 is connected to the flue gas outlet 6 of the regenerator 10, and the lower part of the regenerator 10 is provided with a regenerator gas inlet connected to the regenerator gas distributor 13, to introduce the regenerator gas 4 into the regenerator 10 and distribute it through the regenerator gas distributor 13 to improve the work efficiency of the regenerator 10. The lower part of the regenerator 10 is provided with a regenerated catalyst outlet; the regenerated catalyst outlet is connected to the catalyst second feeding inlet(s) 27 through the regenerator circulation discharge pipe 26; the regenerator circulation discharge pipe 26 is provided with a regenerator circulation discharge pipe control valve 25.

In preferable case, the structural design and parameters of the regenerator gas distributor 13 are the same as those of the raw material second distributor 11. Specifically, the regenerator gas distributor 13 is provided with a regenerator gas main guide pipe extending along the radial direction of the regenerator gas distributor 13, and a plurality of regenerator gas annulus gap guide pipes arranged sequentially along the radial direction of the regenerator gas distributor 13, a regenerator tuyere provided on the regenerator gas annulus gap guide pipe and a regenerator solid guide groove; each regenerator gas annulus gap guide pipe is arranged in an annular shape around the central region of the regenerator gas distributor 13, and the regenerator solid guide groove is located between two adjacent regenerator gas annulus gap guide pipes.

According to the present invention, the regenerator cyclone separator 20 of the regenerator 10 is the same as the settler cyclone separator 19 and is configured as a two-stage or multi-stage cyclone structure connected in series; the gas outlet of the last stage cyclone separator of the regenerator cyclone separator 20 is connected with the flue gas outlet 6 of the regenerator 10, so that the flue gas obtained from the last stage cyclone separator is discharged through the flue gas outlet 6.

In order to obtain a better cooperation of the fluidized bed reactor 7 with the settler 9, as a specific embodiment of the device provided by the present invention, the top of the fluidized bed reactor 7 is provided with a separation riser 15 extending into the settler 9; a riser baffle 14 located above the outlet of the separation riser 15 is provided in the settler 9. Wherein the shape of the riser baffle 14 is herringbone, circular or rectangular, so as to reduce the catalyst particles brought into the settler 9 from the fluidized bed reactor 7.

In the third aspect, the present invention provides a method for preparing low-carbon olefin, which uses the device provided by any of the above technical solutions. The method includes:
the gaseous raw material(s) and the catalyst are reacted in the reaction zone of the fluidized bed reactor 7;
the obtained product and the entrained catalyst are sent to the settler 9 through the top of the reaction zone;
the product and the entrained catalyst are separated by the settler 9, and a part of the separated catalyst is directly supplied into the dense-phase zone 28 through the catalyst first feeding inlet(s) 24, and the other part is regenerated by the regenerator 10 and then fed into the catalyst distribution zone 29 through the catalyst second feeding inlet(s) 27.

According to the present invention, the gaseous raw material(s) may be selected from at least one of methanol, ethanol, propanol, butanol, acetaldehyde, propionaldehyde, butyraldehyde, acetone, butanone, formic acid, acetic acid and propionic acid.

In the method of the present invention, a part of the separated catalyst is fed into the regenerator 10 through the stripper 21, and the stripping medium 3 of the stripper 21 is water vapor.

In the method of the present invention, the pressure in the fluidized bed reactor 7 is 0-0.5 MPa in terms of gauge pressure, the average temperature is 350-560° C., and the temperature difference is <5° C., and the catalyst is SAPO-34, and the linear speed of materials in the dense-phase zone 28 is 1-10 m/s.

In the method of the present invention, among the separated catalysts, the mass ratio of the part supplied to the dense-phase zone 28 to the part supplied to the regenerator 10 is 1:0.2-1.

In the method of the present invention, the coke content of the regenerated catalyst obtained by the regenerator 10 is 5-15% by weight.

In the method of the present invention, the regeneration medium of the regenerator 10 is a mixed gas of CO₂ and air, and the volume ratio of CO₂ to air in the mixed gas is 0.005-0.5:1 to perform partial regeneration reaction to produce a regenerated catalyst; the regeneration temperature of the regenerator 10 is 600-750° C. Introducing CO₂ into the regeneration medium can effectively and selectively eliminate carbon deposits, stably control the carbon deposits of the regenerated catalyst and the temperature of the regenerated catalyst, and at the same time achieve efficient use of reaction heat to convert greenhouse gas CO₂. Among the regenerated catalysts obtained by the regenerator 10, the coke content of a part of the regenerated catalyst is between 1-3% by weight, and the average temperature of the regenerated catalyst is controlled within the range of 400-500° C.

In the method of the present invention, after the regenerated catalyst is regulated by the catalyst distribution components of the catalyst distributor 16, the ratio of the pressure drop generated when the gaseous raw material(s) passes through the dense-phase zone 28 to the pressure drop generated when the gaseous raw material(s) passes through the catalyst distribution zone 29 is 1.5-4:1.

In the method of the present invention, the gaseous raw material(s) passes through the raw material first distributor 8 to perform secondary distribution of the gas direction; the included angle formed by the annulus gap space velocity and the horizontal direction is 45°-75° and the ratio between the internal and external porosity fluctuations is 0.9-0.95:1.

The method of the present invention can achieve uniform distribution of regenerated catalyst and full contact with the raw material(s), effectively suppress uneven temperature distribution and low selection for diene, and can be used in the industrial production of low-carbon olefin.

In one embodiment, the catalyst distribution in the fluidized bed reactor is measured, and the mean square error σ² is used to characterize the heterogeneity of the catalyst. The expression is as shown in formula (1), wherein the larger the value, the more uneven the catalyst distribution in this area; the minimum value 0 means that the concentration at each position is equal to the average concentration value under the ideal state. Wherein, σ₁² represents the concentration distribution of the regenerated catalyst relative to the three phases (gas phase, circulating catalyst, regenerated catalyst);

$${σ}_{i}^{2} = \sum {( {C}_{i} / \overline{C} - 1 )}^{2} / m \qquad ( I )$$

$${c}_{1} = {c}_{re} \qquad ( II )$$

wherein, cᵣₑ refers to the particle concentration of the regenerated catalyst in each grid area required for calculation; c̅ is the particle concentration of all catalysts in each grid area; m represents the number of the measured reactor area cross-sections, in different measurements generally is 5≤m≤50; i represents the type of catalyst fed to the reactor.

For example, in the present invention, the fluidized bed reactor 7 preferably includes a catalyst first feeding inlet(s) 24 and a catalyst second feeding inlet(s) 27 to feed different catalysts, such as circulating catalysts and regenerated catalysts respectively; correspondingly, in this case, i=2, c₁=cᵣₑ, wherein cᵣₑ refers to the particle concentration of the regenerated catalyst in each grid area required for the calculation, and c₂=c_{circulate}, where c_{circulate} refers to the particle concentration of circulating catalyst in each grid area required for calculation.

It is known to those skilled in the art that the data of each grid point of each section can be obtained according to CFD calculation, and then be extracted for data processing, thereby obtaining the mean square error data.

A relatively preferred specific example of the device and method for preparing low-carbon olefin described in the present invention will be described below based on the device for preparing low-carbon olefin shown in FIG. 1:
the device for preparing low-carbon olefin includes a fluidized bed reactor 7, a settler 9 and a regenerator 10;
the reaction zone of the fluidized bed reactor 7 is provided with a raw material first distributor 8, a raw material second distributor 11 and a catalyst distributor 16 in order from bottom to top; a dense-phase region 28 of the catalyst is formed between the raw material first distributor 8 and the raw material second distributor 11; the region where the catalyst distributor 16 is located forms a catalyst distribution zone 29 connected with the dense-phase region 28; a plurality of catalyst first feeding inlet(s) 24 are provided on the side wall of reactor in the dense-phase region 28;
the raw material first distributor 8 includes a first distributor central region 40 and a first distributor external annular region 42 located on the outer periphery of the first distributor central region 40; the first distributor external annular region 42 is provided with the first distributor enhancement region 38 corresponding to the catalyst first feeding inlet(s) 24; the catalyst first feeding inlet(s) 24 is located above the center of the outer edge of its corresponding the first distributor enhancement region 38. The first distributor enhancement region 38 is provided with a plurality of columnar first distributor enhanced nozzles 39; the included angle formed by the center line of the first distributor enhanced nozzle 39 and the horizontal direction is 45°-75°. The first distributor enhanced nozzle 39 includes enhanced nozzle inlets 39-1, enhanced nozzle reducing pipe 39-2, enhanced nozzle pipe throat 39-3, enhanced nozzle expansion section 39-4 and enhanced nozzle outlet 39-5, which are sequentially connected; enhanced nozzle inlet 39-1 is connected to the main body of the raw material first distributor 8;
the raw material second distributor 11 is provided with a second distributor gas main guide pipe 43 extending along the radial direction of the raw material second distributor 11, a plurality of second distributor gas annulus gap guide pipes 44 arranged sequentially along the radial direction of the raw material second distributor 11, the second distributor tuyere 45 provided on the second distributor gas annulus gap guide pipes 44 and the second distributor solid guide groove 46; each second distributor gas annulus gap guide pipe 44 is arranged to be distributed in an annular shape around the central region of the raw material second distributor 11; The second distributor solid guide groove 46 is located between the two adjacent second distributor gas annulus gap guide pipes 44; and the ratio of the width of the second distributor gas annulus gap guide pipe 44 to the width of the second distributor solid guide groove 46 is 1:2-6;
the catalyst distributor 16 includes a catalyst distributor main guide pipe 47 and multiple layers of catalyst distribution components 48 distributed along the up and down direction of the catalyst distributor main guide pipe 47; the catalyst distributor main guide pipe 47 is arranged vertically in the reaction zone and is connected to the catalyst second feeding inlet(s) 27; and the catalyst distribution components 48 includes a plurality of catalyst first distribution conduits 49 and a plurality of catalyst second distribution conduits 50. The catalyst first distribution conduit(s) 49 and the catalyst second distribution conduit(s) 50 are distributed circumferentially and staggered along the catalyst distributor main guide pipe 47 and are both connected with the catalyst distributor main guide pipe 47. The catalyst first distribution conduit(s) 49 and the catalyst second distribution conduits 50 are respectively provided with a plurality of catalyst outlets 51. The length of the catalyst distribution pipes corresponding to the catalyst distribution components 48 decreases from top to bottom; and the plurality of catalyst outlets 51 are equidistantly distributed on the catalyst first distribution conduit(s) 49 and the catalyst second distribution conduit(s) 50;
the fluidized bed reactor 7 is provided with a raw material first feeding inlet(s) 1 and a raw material second feeding inlet(s) 2. The raw material first feeding inlet(s) 1 is connected to the bottom of the raw material first distributor 8; the raw material second feeding inlet(s) 2 is located at the intersection area of the dense-phase region 28 and the catalyst distribution zone 29, the ratio of the feeding amount of the raw material first feeding inlet(s) 1 to the feeding amount of the raw material second feeding inlet(s) 2 is 1-10:1;
a circulating distribution baffle 34 connected to the inner wall of the fluidized bed reactor is provided above the catalyst first feeding inlet(s) 24; a plurality of circulating distribution baffle grooves 37 are provided on the circulating distribution baffle 34, and the angle α formed between the circulating distribution baffle grooves 37 and the horizontal direction is 30°-75°; the settler 9 includes a lower section of settler 17 located at lower part, an upper section of settler 18 located at upper part, and a settler cyclone separator 19 located in the upper section of settler 18; the gas outlet of the settler cyclone separator 19 is connected to the product gas outlet 5 of the settler 9, the lower part of the lower section 17 of the settler is provided with a settler distribution plate 12, the upper part of the settler distribution plate 12 is connected to the corresponding catalyst first feeding inlet(s) 24 through a circulation pipe(s) 22, and a circulation pipe control valve(s) 23 is provided on the circulation pipe(s) 22. The upper part of the settler distribution plate 12 is connected to the stripper 21 through the stripper feed pipe 30; and the upper section of settler 18 is hemispherical. The settler distribution plate 12 is provided with the settler first distribution plate holes 35 and the settler second distribution plate holes 36, the settler first distribution plate holes 35 and the settler second distribution plate holes 36 are respectively arranged to be annularly distributed around the central region of the settler distribution plate 12;
the outlet of the stripper 21 is connected to the regenerator feed pipe 33 of the regenerator 10 through the stripper discharge pipe 31, and a stripper control valve 32 is provided on the stripper discharge pipe 31;
the regenerator 10 is provided with a regenerator gas distributor 13 and a regenerator cyclone separator 20 located above the regenerator gas distributor 13. The gas outlet of the regenerator cyclone separator 20 is connected with the flue gas outlet 6 of the regenerator 10. The structural design and parameters of the regenerator gas distributor 13 are the same as those of the raw material second distributor 11. The lower part of the regenerator 10 is provided with a regenerated catalyst outlet, and the regenerated catalyst outlet is connected to the second inlet of catalyst 27 through the regenerator circulation discharge pipe 26;
the top of the fluidized bed reactor 7 is provided with a separation riser 15 extending into the settler 9, and a riser baffle 14 located above the outlet of the separation riser 15 is provided in the settler 9.

Using the above relatively preferred specific example of the device for preparing low-carbon olefin, the specific steps of the method for preparing low-carbon olefin are:
S1. the gaseous raw material(s) and catalyst from the raw material first feeding inlet(s) 1 and the raw material second feeding inlet(s) 2 are transported to the raw material first distributor 8 and the raw material second distributor 11, and a part of them is distributed to the dense-phase zone 28 through the first distributor enhanced nozzle 39 and the tuyere 41 in the central region of the first distributor, and the other part is distributed to the catalyst distribution zone 29 through the raw material second distributor 11, so that the gaseous raw material(s) and catalyst are reacted in the reaction zone of the fluidized bed reactor 7;
S2. let the product obtained by the reaction described in step S1 and the entrained catalyst enter the settler 9 from the separation riser 15, the product and the entrained catalyst are separated by the action of the settler cyclone separator 19, and the obtained product gas is discharged through the product gas outlet 5, a part of the separated catalyst is transported from the circulation pipe(s) 22 to the catalyst first feeding inlet(s) 24 to be directly supplied into the dense-phase zone 28, and let the other part enter the stripper 21 through the stripper feed pipe 30; after being stripped by the stripper 21, it is transported to the regenerator 10 through the stripper discharge pipe 31 and the regenerator feed pipe 33; the flue gas and the regenerated catalyst are obtained after being separated and regenerated by the regenerator cyclone separator 20, the flue gas is discharged through the flue gas outlet 6, and the regenerated catalyst is input into the catalyst second feeding inlet(s) 27 through the regenerator circulation discharge pipe 26 to be supplied into the catalyst distribution zone 29, and to be contacted and reacted with the gaseous raw material(s) in the dense-phase zone 28.

The invention thus provides the following second series of exemplary embodiments:
1. A fluidized bed reactor, characterized in that the reaction zone of the fluidized bed reactor is provided with a raw material first distributor (8), a raw material second distributor (11) and a catalyst distributor (16) in sequence from bottom to top, the catalyst distributor (16) is connected with the second catalyst feed pipe (27), and a dense-phase zone (28) is formed between the raw material first distributor (8) and the raw material second distributor (11), the area where the catalyst distributor (16) is located is formed as a catalyst distribution zone (29) connected with the dense-phase zone (28), and at least one catalyst first feeding inlet(s) (24) is provided on the side wall of the reactor of the dense-phase zone (28).
2. The fluidized bed reactor according to the second series of exemplary embodiment 1, characterized in that the raw material first distributor (8) includes a first distributor central region (40) and a first distributor external region (42) located on the outer periphery of the central region (40) of the first distributor, the first distributor external region (42) is provided with a first distribution enhancement region (38) corresponding to the catalyst first feeding inlet(s) (24).
3. The fluidized bed reactor according to the second series of exemplary embodiment 2, characterized in that the catalyst first feeding inlet(s) (24) is located above the center of the outer edge of its corresponding first distributor enhancement region (38).
4. The fluidized bed reactor according to the second series of exemplary embodiment 2, characterized in that, the first distributor enhancement region (38) is provided with a plurality of columnar first distributor enhanced nozzles (39), and the included angle formed by the center line of the first distributor enhanced nozzle(s) (39) and the horizontal direction is 45°-75°.
5. The fluidized bed reactor according to the second series of exemplary embodiment 4, characterized in that the first distributor enhanced nozzle(s) (39) includes an enhanced nozzle inlet (39-1), an enhanced nozzle reducing pipe (39-2), an enhanced nozzle pipe throat (39-3), an enhanced nozzle expansion section (39-4) and the enhanced nozzle outlet (39-5), which are connected in sequence; the enhanced nozzle inlet (39-1) is connected to the main body of the raw material first distributor (8).
6. The fluidized bed reactor according to the second series of exemplary embodiment 5, characterized in that the ratio of the diameter of the enhanced nozzle pipe throat (39-3) to the diameter of the enhanced nozzle inlet (39-1) is 1:5-20, and the ratio of the length of the enhanced nozzle pipe throat (39-3) to the diameter of the enhanced nozzle pipe throat (39-3) is 5-10:1.
7. The fluidized bed reactor according to any one of the second series of exemplary embodiments 1 to 6, characterized in that the raw material second distributor (11) is provided with the second distributor gas main guide pipe (43) radially extending along the radial direction of the raw material second distributor (11), and a plurality of second distributor gas annulus gap guide pipes (44) arranged sequentially along the radial direction of the raw material second distributor (11), and the second distributor air outlet (45) provided on the second distributor gas annulus gap guide pipe (44) and a second distributor solid guide groove(s) (46), each of the second distributor gas annulus gap guide pipe (44) is arranged to be annularly distributed around the central region of the raw material second distributor (11), and the second distributor solid guide groove (46) is located between two adjacent the second distributor gas annulus gap guide pipes (44).
8. The fluidized bed reactor according to the second series of exemplary embodiment 7, characterized in that the ratio of the width of the second distributor gas annulus gap guide pipe (44) to the width of the second distributor solid guide groove (46) is 1:2-6.
9. The fluidized bed reactor according to any one of the second series of exemplary embodiments 1 to 6, characterized in that the catalyst distributor (16) includes a catalyst distributor main guide pipe (47) and a multi-layers of catalyst distribution system (48) distributed along the up and down direction of the catalyst distributor main guide pipe (47); the catalyst distributor main guide pipe (47) is arranged vertically in the reaction zone and connected with the second catalyst feed pipe (27), and the catalyst distribution system (48) includes a plurality of catalyst first distribution conduits (49) and a plurality of catalyst second distribution conduits (50), the catalyst first distribution conduit(s) (49) and the catalyst second distribution conduit(s) (50) are distributed circumferentially and staggered along the catalyst distributor main conduit (47) and are all connected with the catalyst distributor main conduit (47); the catalyst first distribution conduit(s) (49) and the catalyst second distribution conduit(s) (50) are respectively provided with a plurality of catalyst outlets (51).
10. The fluidized bed reactor according to the second series of exemplary embodiments 9, characterized in that the lengths of the catalyst first distribution conduit(s) (49) and the catalyst second distribution conduit(s) (50) corresponding to the catalyst distribution system (48) decrease sequentially from top to bottom, and the catalyst outlets (51) on the catalyst first distribution conduit(s) (49) and the catalyst second distribution conduit(s) (50) are equidistantly distributed.
11. The fluidized bed reactor according to any one of the second series of exemplary embodiments 1 to 6, characterized in that the fluidized bed reactor is provided with a first feeding inlet(s) (1) and a second feeding inlet(s) (2), the first feeding inlet(s) (1) is connected to the bottom of the raw material first distributor (8), and the second feeding inlet(s) (2) is located in the intersection area of the dense-phase zone (28) with the catalyst distribution zone (29).
12. The fluidized bed reactor according to the second series of exemplary embodiment 11, characterized in that the ratio of the feeding amount of the first feeding inlet(s) (1) to the feeding amount of the second feeding inlet(s) (2) is 1-10:1.
13. The fluidized bed reactor according to any one of the second series of exemplary embodiments 1 to 6, characterized in that a circulating distribution baffle (34) connected to the inner wall of the fluidized bed reactor is provided above the catalyst first feeding inlet(s) (24).
14. The fluidized bed reactor according to the second series of exemplary embodiment 13, characterized in that the ratio of the distance between the circulating distribution baffle (34) and the catalyst first feeding inlet(s) (24) to the hole diameter of the catalyst first feeding inlet(s) (24) is 1-10:1.
15. The fluidized bed reactor according to the second series of exemplary embodiment 13, characterized in that the circulating distribution baffle (34) is provided with a plurality of circulating distribution baffle grooves (37), and the angle (a) formed by the circulating distribution baffle grooves (37) and the horizontal direction is 30°-75°.
16. A device for preparing low-carbon olefin, characterized in that the device includes a fluidized bed reactor (7) according to any one of the second series of exemplary embodiments 1 to 15, a settler (9) and regenerator (10), the settler (9) is connected to the upper part of the reaction zone of the fluidized bed reactor (7), and the lower part of the settler (9) is connected to the catalyst first feeding inlet(s) (24) and the regenerator (10) respectively, and the regenerated catalyst outlet of the regenerator (10) is connected to the second catalyst feed pipe (27).
17. The device according to the second series of exemplary embodiment 16, characterized in that the number of the catalyst first feeding inlet(s) (24) is an even number, and the catalyst first feeding inlet(s) (24) are symmetrically arranged along the central axis of the fluidized bed reactor (7), and the lower part of the settler (9) is connected to the catalyst first feeding inlet(s) (24) through a circulation pipe(s) (22).
18. The device according to the second series of exemplary embodiment 17, characterized in that a lower section of settler (17), an upper section of settler (18) located above the lower section of settler (17), and a settler cyclone separator (19) located on the upper section of settler (18) are provided in the settler (9); the gas outlet of the settler cyclone separator (19) is connected with the product gas outlet (5) of the settler (9); a settler distribution plate (12) is provided at the lower part of the lower section of settler (17), the upper part of the settler distribution plate (12) is connected to the circulation pipe(s) (22), and is connected to the regenerator (10) through the stripper (21).
19. The device according to the second series of exemplary embodiment 18, characterized in that the top of the upper section of settler (18) is hemispherical, the settler distribution plate (12) is provided with a settler first distribution plate holes (35) and a settler second distribution plate holes (36), and the settler first distribution plate holes (35) and the settler second distribution plate holes (36) are respectively arranged to be annularly distributed around the central area of the settler distribution plate (12), and the size ratio of the settler first distribution plate holes (35) to the settler second distribution plate holes (36) is 1-3:4.
20. The device according to any one of the second series of exemplary embodiments 16 to 19, characterized in that a regenerator gas distributor (13) and a regenerator cyclone separator (20) located above the regenerator gas distributor (13) are provided in the regenerator (10), the gas outlet of the regenerator cyclone separator (20) is connected with the flue gas outlet (6) of the regenerator (10);
the regenerator gas distributor (13) is provided with a regenerator gas main guide pipe extending along the radial direction of the regenerator gas distributor (13), a plurality of regenerator gas annulus gap guide pipes arranged sequentially along the radial direction of the regenerator gas distributor (13), a regenerator tuyere arranged on the regenerator gas annulus gap guide pipe and a regenerator solid guide groove, each of the regenerator gas annulus gap guide pipes are arranged to be distributed annularly around the central region of the regenerator gas distributor (13), and the regenerator solid guide groove is located between two adjacent regenerator gas annulus gap guide pipes.
21. The device according to any one of the second series of exemplary embodiments 16 to 19, characterized in that the top of the fluidized bed reactor (7) is provided with a separation riser (15) extending into the settler (9), the settler (9) is provided with a baffle (14) located above the outlet of the separation riser (15).
22. A method for preparing low-carbon olefin using the device according to the second series of exemplary embodiment 16, the method comprising:
the gaseous raw material(s) and catalyst are reacted in the reaction zone of the fluidized bed reactor;
the obtained product and the entrained catalyst are introduced to a settler through the top of the reaction zone;
the product and the entrained catalyst are separated by the settler, and a part of the separated catalyst is directly supplied into the dense-phase zone through the catalyst first feeding inlet, and the other part is fed into the catalyst distribution zone from the second catalyst feed pipe after being regenerated by the regenerator.
23. The method according to the second series of exemplary embodiments 22, characterized in that a part of the separated catalyst is supplied into the regenerator through a stripper, and the stripping medium of the stripper is water steam.
24. The method according to the second series of exemplary embodiment 22, characterized in that the pressure in the fluidized bed reactor is 0-0.5 MPa in gauge pressure, the average temperature is 350-560° C., and the temperature difference is <5° C., the catalyst is SAPO-34, and the material linear speed in the dense-phase zone is 1-10 m/s.
25. The method according to the second series of exemplary embodiment 22, characterized in that, among the separated catalysts, the mass ratio of the part fed into the dense-phase zone to the part fed into the regenerator is 1:0.2-1.
26. The method according to the second series of exemplary embodiment 22, characterized in that the coke content of the regenerated catalyst obtained by the regenerator is 5-15% by weight.
27. The method according to the second series of exemplary embodiment 22, characterized in that the regeneration medium of the regenerator is a mixed gas of CO₂ and air, and the volume ratio of CO₂ to air in the mixed gas is 0.005-0.5:1; the regeneration temperature of the regenerator is 600-750° C.
28. The method according to the second series of exemplary embodiment 22, characterized in that the ratio of the pressure drop generated by the gaseous raw material(s) when passing through the dense-phase zone to the pressure drop generated by the gaseous raw material(s) when passing through the catalyst distribution zone is 1.5-4:1.
29. The method according to the second series of exemplary embodiment 22, characterized in that after the gaseous raw material(s) passes through the raw material first distributor, the included angle formed by the annulus gap space velocity and the horizontal direction is 45°-75°, the ratio between the internal and external porosity fluctuations is 0.9-0.95:1.

The present invention will be further described below through examples, but the protection scope of the present invention is not limited thereto.

In the following examples, the experimental methods used are conventional methods unless otherwise specified; the materials, reagents, etc. used can be obtained from commercial sources unless otherwise specified.

### Example 1

The relatively preferred specific example of the above-mentioned device for preparing low-carbon olefin and the specific steps of the method for preparing low-carbon olefin were used to prepare low-carbon olefin;

Referring to FIG. 1, wherein, there were four catalyst first feeding inlets 24 in the device for preparing low-carbon olefin; the structure of the raw material first distributor 8 was as shown in FIGS. 3 and 4, and the opening ratio was 1%. The included angle formed by the enhanced nozzle reducing pipe 39-2 and the horizontal direction was 45°, the included angle formed by the enhanced nozzle expansion section 39-4 and the horizontal direction was 70°, and the ratio of the diameter of the enhanced nozzle pipe throat 39-3 to the diameter of the enhanced nozzle inlet 39-1 was 1:10, and the ratio of the length of the enhanced nozzle pipe throat 39-3 to the diameter of the enhanced nozzle pipe throat 39-3 was 8:1. The first distributor enhancement region 38 has an opening ratio of 0.2% and a hole diameter of 2 mm. The opening ratio of the first distributor external region 42 and the opening ratio of the first distributor central region 40 were both 2% and the hole diameters thereof were both 6 mm. The first distributor central region 40 was a circle with a radius r, and the first distributor external region 42 was an annulus with the difference between the outer diameter and the inner diameter being d, r/d=½, and the ratio of their areas was ½, the inner diameter D of the reactor was 8 meters.

For the raw material second distributor 11, the ratio of the width of the second distributor gas annulus gap guide pipe 44 to the width of the second distributor solid guide groove 46 was 1:4. The raw material second distributor 11 has an opening ratio of 5% and was ½ axially away from the raw material first distributor 8.

The catalyst distribution components 48 adopts a dendritic arrangement scheme and was arranged in 3 layers, each layer was provided with 3 catalyst distribution conduits (the catalyst first distribution conduit 49 and the catalyst second distribution conduit 50 were distributed in staggered manner); the diameter size of the effective channel of the catalyst outlet 51 was 60 mm, and the ratio of the center distance of two adjacent catalyst outlets 51 to the width of the catalyst outlet 51 along their distribution direction was 3:1. The height of the catalyst distributor main guide pipe 47 upward from the raw material first distributor (8) was ¾h; the lengths of the multi-layer distribution pipes were 0.75D, 0.75²D and 0.75³ D respectively.

Two raw material feeding inlets were used, wherein the feed amount ratio of the raw material first feeding inlet(s) 1 to the raw material second feeding inlet(s) 2 was 6:1, and the ratio of the distance between the circulating distribution baffle 34 and the catalyst first feeding inlet(s) 24 to the hole diameter of the catalyst first feeding inlet(s) 24 was 6:1, the angle α formed between the circulating distribution baffle groove 37 and the horizontal direction was 55°, the ratio of the size of the settler first distribution plate holes 35 to the size of the settler second distribution plate holes 36 was 2:4, and the settler cyclone separator 19 and the regenerator cyclone separator 20 were respectively arranged in a two-stage series cyclone separators configuration.

In the process of preparing low-carbon olefin, methanol with a purity of 99.5% was used as the raw material, SAPO-34 was used as the catalyst, and the stripping medium 3 of the stripper 21 was water vapor. The pressure in the fluidized bed reactor 7 was 0.3 MPa, the average temperature was 450° C., the temperature difference was controlled to be <5° C., and the linear speed was 5 m/s. The mass ratio of the catalyst supplied to the dense-phase zone 28 through the catalyst first feeding inlet(s) 24 to the catalyst supplied to the regenerator 10 through the catalyst second feeding inlet(s) 27 was 1:0.5. The coke content of the regenerated catalyst obtained by the regenerator 10 was 10% by weight. The regeneration medium of the regenerator 10 was a mixed gas of CO₂ and air, and the volume ratio of CO₂ to air in the mixture was 0.2:1. The regeneration temperature of regenerator 10 was 650° C. The ratio of the pressure drop generated when the gaseous raw material(s) passes through the dense-phase zone 28 to the pressure drop generated when the gaseous raw material(s) passes through the catalyst distribution zone 29 was 3:1. After the gaseous raw material(s) passes through the raw material first distributor 8, the included angle formed by the annulus gap space velocity and the horizontal direction was 45°, and the ratio between the internal and external porosity fluctuations was 0.95:1.

The variation of regenerated catalyst distribution unevenness with height was shown in FIG. 13. The three-phase average uniformity of the regenerated catalyst was 0.29. The methanol conversion rate was 99.995%, and the total yield (mass) of ethylene and propylene was 84.8%.

### Example 2

This example was conducted according to the device and method steps of Example 1, except that:
two catalyst first feeding inlets 24 were provided in the device for preparing low-carbon olefin. The structure of the raw material first distributor 8 was as shown in FIG. 2. The included angle range formed between the enhanced nozzle reducing pipe 39-2 and the horizontal direction was 45°, the included angle formed by the enhanced nozzle expansion section 39-4 and the horizontal direction was 70°, and the ratio of the diameter of the enhanced nozzle pipe throat 39-3 to the diameter of the enhanced nozzle inlet 39-1 was 1:5, and the ratio of the length of the enhanced nozzle pipe throat 39-3 to the diameter of the enhanced nozzle pipe throat 39-3 was 5:1. The ratio of the width of the second distributor gas annulus gap guide pipe 44 to the width of the second distributor solid guide groove 46 was 1:2. The catalyst distribution components 48 was arranged in 3 layers, and each layer was provided with 2 catalyst distribution conduits (the catalyst first distribution conduit 49 and the catalyst second distribution conduit 50 were distributed in staggered manner), the diameter of the effective channel of the catalyst outlet 51 was 80 mm. The size ratio of the center distance of the two adjacent catalyst outlets 51 to the width of the catalyst outlet 51 along their distribution direction was 1.5:1. The feed amount ratio of the raw material first feeding inlet(s) 1 to the raw material second feeding inlet(s) 2 was 2:1, and the ratio of the distance between the circulating distribution baffle 34 and the catalyst first feeding inlet(s) 24 to the hole diameter of the catalyst first feeding inlet(s) 24 was 2:1, the angle α formed by the circulating distribution baffle groove 37 and the horizontal direction was 30°, and the size ratio of the settler first distribution plate holes 35 and the settler second distribution plate holes 36 was 1:4. The settler cyclone separator 19 and the regenerator cyclone separator 20 were respectively arranged in a two-stage series cyclone separators configuration.

In the process of preparing low-carbon olefin, the raw material was methanol with a purity of 99.5%, the catalyst was SAPO-34, the pressure in the fluidized bed reactor 7 was 0.1 MPa, the average temperature was 350° C., the temperature difference was <5° C., and the line speed was 1 m/s, and the mass ratio of the catalyst supplied into the dense-phase zone 28 through the catalyst first feeding inlets 24 to the catalyst supplied into the regenerator 10 through the catalyst second feeding inlet(s) 27 was 1:0.2. The stripping medium 3 of the stripper 21 was water vapor. The coke content of the regenerated catalyst obtained by the regenerator 10 was 5% by weight. The regeneration medium of the regenerator 10 was a mixed gas of CO₂ and air. The volume ratio of CO₂ to air in the mixed gas was 0.01:1. The regeneration temperature of the regenerator 10 was 600° C. The ratio of the pressure drop generated when the gaseous raw material(s) passes through the dense-phase zone 28 to the pressure drop generated when the gaseous raw material(s) passes through the catalyst distribution zone 29 was 1.5:1. After the gaseous raw material(s) passes through the raw material first distributor 8, the included angle formed by the annulus gap space velocity and the horizontal direction was 45°-75°, and the ratio between the internal and external porosity fluctuations was 0.9-0.95:1.

The three-phase average uniformity of the regenerated catalyst was 0.89. The methanol conversion rate was 99.985%, and the total yield (mass) of ethylene and propylene was 83.58%.

### Example 3

This example was conducted according to the device and method steps of Example 1, except that: two catalyst first feeding inlets 24 were provided in the device for preparing low-carbon olefin, and the structure of the raw material first feed distributor 8 was as shown in FIG. 2; the included angle range formed between the enhanced nozzle reducing pipe 39-2 and the horizontal direction was 45°, the included angle formed by the enhanced nozzle expansion section 39-4 and the horizontal direction was 70°, and the ratio of the diameter of the enhanced nozzle pipe throat 39-3 to the diameter of the enhanced nozzle inlet 39-1 was 1:20, and the ratio of the length of the enhanced nozzle pipe throat 39-3 to the diameter of the enhanced nozzle pipe throat 39-3 was 10:1. The ratio of the width of the second distributor gas annulus gap guide pipe 44 to the width of the second distributor solid guide groove 46 was 1:6. The catalyst distribution components 48 was arranged in 3 layers, and each layer was provided with 2 catalyst distribution conduits (the catalyst first distribution conduit 49 and the catalyst second distribution conduit 50 were distributed in staggered manner). The diameter of the effective channel of the catalyst outlet 51 was 100 mm, the size ratio of the center distance of the two adjacent catalyst outlets 51 to the width of the catalyst outlet 51 along their distribution direction was 5:1. The feed amount ratio of the raw material first feeding inlet(s) 1 to the raw material second feeding inlet(s) 2 was 10:1, and the ratio of the distance between the circulating distribution baffle 34 and the catalyst first feeding inlet(s) 24 to the hole diameter of the catalyst first feeding inlet(s) 24 was 10:1, the angle α formed by the circulating distribution baffle groove 37 and the horizontal direction was 75°, and the size ratio of the settler first distribution plate holes 35 and the settler second distribution plate holes 36 was 3:4; the settler cyclone separator 19 and the regenerator cyclone separator 20 were respectively arranged in a two-stage series cyclone separators configuration.

In the process of preparing low-carbon olefin, the raw material was methanol with a purity of 99.5%, the catalyst was SAPO-34, the pressure in the fluidized bed reactor 7 was 0.5 MPa, the average temperature was 560° C., the temperature difference was <5° C., and the line speed was 10 m/s; and the mass ratio of the catalyst supplied into the dense-phase zone 28 through the catalyst first feeding inlet(s) 24 to the catalyst supplied into the regenerator 10 through the catalyst second feeding inlet(s) 27 was 1:1. The stripping medium 3 of the stripper 21 was water vapor. The coke content of the regenerated catalyst obtained by the regenerator 10 was 15% by weight. The regeneration medium of the regenerator 10 was a mixed gas of CO₂ and air. The volume ratio of CO₂ to air in the mixed gas was 0.5:1. The regeneration temperature of regenerator 10 was 750° C. The ratio of the pressure drop generated when the gaseous raw material(s) passes through the dense-phase zone 28 to the pressure drop generated when the gaseous raw material(s) passes through the catalyst distribution zone 29 was 4:1. After the gaseous raw material(s) passes through the raw material first distributor 8, the included angle formed by the annulus gap space velocity and the horizontal direction was 45°-75°, and the ratio between the internal and external porosity fluctuations was 0.9-0.95:1.

The methanol conversion rate was 99.981%, and the total yield (mass) of ethylene and propylene was 83.14%.

### Example 4

This example was conducted according to the device and method steps of Example 1, except that: the raw material first distributor 8 was only provided with the first distributor central region tuyere 41, and is not provided with the first distributor enhancement region 38 and the first distributor enhancement nozzle 39 corresponding to the catalyst first feeding inlet(s) 24.

The methanol conversion rate was 99.98%, and the total yield (mass) of ethylene and propylene was 82.65%.

### Example 5

This example was conducted according to the device and method steps of Example 1, except that: the included angle formed by the enhanced nozzle reducing pipe 39-2 on the raw material first distributor 8 and the horizontal direction was 90°, and the included angle formed by the enhanced nozzle expanded section 39-4 and the horizontal direction was 90°.

The methanol conversion rate was 99.96%, and the total yield (mass) of ethylene and propylene was 80.61%.

### Example 6

This example was conducted according to the device and method steps of Example 1, except that: the raw material second distributor 11 was only provided with a plurality of openings.

The methanol conversion rate was 99.959%, and the total yield (mass) of ethylene and propylene was 80.68%.

### Example 7

This example was conducted according to the device and method steps of Example 1, except that: the ratio of the width of the second distributor gas annulus gap guide pipe 44 to the width of the second distributor solid guide groove 46 was 2:1.

The methanol conversion rate was 99.978%, and the total yield (mass) of ethylene and propylene was 80.61%.

### Example 8

This example was conducted according to the device and method steps of Example 1, except that: the catalyst distributor 16 includes a catalyst distributor main guide pipe 47, and the catalyst distributor main guide pipe 47 was arranged vertically in the reaction zone and connected with the catalyst second feeding inlet(s) 27; the catalyst distributor main guide pipe 47 was provided with a plurality of vertically arranged openings.

The methanol conversion rate was 99.977%, and the total yield (mass) of ethylene and propylene was 82.45%.

### Example 9

This example was conducted according to the device and method steps of Example 1, except that: the raw material first feeding inlet(s) 1 and the raw material second feeding inlet(s) 2 were both connected to the bottom of the raw material first distributor 8.

The methanol conversion rate was 99.95%, and the total yield (mass) of ethylene and propylene was 81.7%.

### Example 10

This example was conducted according to the device and method steps of Example 1, except that: the ratio of the feeding amount of the raw material first feeding inlet(s) 1 to the feeding amount of the raw material second feeding inlet(s) 2 was 1:5.

The methanol conversion rate was 99.975%, and the total yield (mass) of ethylene and propylene was 82.75%.

### Example 11

This example was conducted according to the device and method steps of Example 1, except that: no circulating distribution baffle 34 was provided above the catalyst first feeding inlet(s) 24.

The methanol conversion rate was 99.945%, and the total yield (mass) of ethylene and propylene was 81.71%.

### Example 12

This example was conducted according to the device and method steps of Example 1, except that: the circulating distribution baffle 34 was not provided with a circulating distribution baffle groove 37.

The methanol conversion rate was 99.94%, and the total yield (mass) of ethylene and propylene was 81.63%.

### Example 13

This example was conducted according to the device and method steps of Example 1, except that: the settler distribution plate 12 was only provided with a plurality of settler first distribution plate holes 35.

The methanol conversion rate was 99.965%, and the total yield (mass) of ethylene and propylene was 82.5%.

### Example 14

This example was conducted according to the device and method steps of Example 1, except that: the regeneration medium of the regenerator 10 was air.

The methanol conversion rate was 99.974%, and the total yield (mass) of ethylene and propylene was 81.55%.

### Example 15

This example was conducted according to the device and method steps of Example 1, except that: the regeneration medium of the regenerator 10 was a mixed gas of CO₂ and air, and the volume ratio of CO₂ to air in the mixed gas was 0.7:1.

The methanol conversion rate was 99.975%, and the total yield (mass) of ethylene and propylene was 82.53%.

### Comparative Example 1

This comparative example was conducted according to the device and method steps of Example 1, except that: the raw material first distributor 8, the raw material second distributor 11 and the catalyst distributor 16 were not provided in the fluidized bed reactor 7. Only the catalyst first feeding inlet(s) 24 and the catalyst second feeding inlet(s) 27 were respectively connected to the reaction zone of the fluidized bed reactor 7.

The methanol conversion rate was 99.91%, and the total yield (mass) of ethylene and propylene was 80.05%.

### Comparative Example 2

This comparative example was conducted according to the device and method steps of Example 1, except that: only the raw material first distributor 8 was provided in the fluidized bed reactor 7, and the raw material second distributor 11 and the catalyst distributor 16 were not provided in the fluidized bed reactor 7. The catalyst first feeding inlet(s) 24 and the catalyst second feeding inlet(s) 27 were respectively connected to the top of the raw material first distributor 8. The fluidized bed reactor 7 was provided with a raw material first feeding inlet(s) 1 connected to the bottom of the raw material first distributor 8.

The methanol conversion rate was 99.93%, and the total yield (mass) of ethylene and propylene was 81.01%.

### Comparative Example 3

This comparative example was conducted according to the device and method steps of Example 1, except that: the raw material first distributor 8 and the raw material second distributor 11 were provided in the fluidized bed reactor 7; however, the catalyst distributor 16 was not provided in the fluidized bed reactor 7, but the regenerated catalyst was fed from the side as usual. The catalyst first feeding inlet(s) 24 and the catalyst second feeding inlet(s) 27 were respectively connected to the top of the raw material first distributor 8. The fluidized bed reactor 7 was provided with a raw material first feeding inlet(s) 1 connected to the bottom of the raw material first distributor 8.

The methanol conversion rate was 99.84%, and the total yield (mass) of ethylene and propylene was 78.5%.

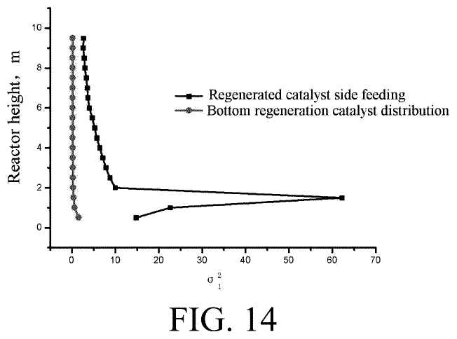

A comparison of the variation of the unevenness of the regenerated catalyst distribution with height in Comparative Example 3 and Example 1 was shown in FIG. 14. It was clear that the range of the uniformity distribution variation of Comparative Example 3 wherein the regenerated catalyst was fed from the side was 2.57-62.15; by contrast, the range of the uniformity distribution variation of Example 1 using the bottom regenerated catalyst distributor 16 was 0.16-1.56, the uniformity was significantly improved.

The preferred embodiments of the present invention have been described in detail above, however, the present invention is not limited thereto. Within the scope of the technical concept of the present invention, many simple modifications can be made to the technical solution of the present invention, including the combination of various technical features in any other suitable manner. These simple modifications and combinations should also be regarded as the disclosed contents of the present invention, and all belong to the protection scope of the present invention.

1. A fluidized bed reactor, including:
a raw material first distributor (8), a raw material second distributor (11) and a catalyst distributor (16) in the reaction zone of the fluidized bed reactor, which are used to distribute gaseous raw material(s), wherein the raw material first distributor (8) is below the raw material second distributor (11); wherein the raw material first distributor (8) and the raw material second distributor (11) have same or different opening ratio, each independently is 0.05-5%;
a raw material first feeding inlet(s) (1) at the bottom of the fluidized bed reactor, which allows at least a part of the raw material(s) to be distributed twice sequentially through the raw material first distributor (8) and the raw material second distributor (11);
the catalyst distributor (16) includes a catalyst distributor main guide pipe (47), which is coaxially distributed with the raw material first distributor (8) and the raw material second distributor (11), wherein the catalyst distributor main guide pipe (47) passes through the raw material first distributor (8) and the raw material second distributor (11) from bottom to top.

2. The fluidized bed reactor according to claim 1, characterized in that the fluidized bed reactor has one or more raw material Y-th feeding inlet(s) sequentially arranged from bottom to top above the raw material first feeding inlet(s) (1), wherein Y is a positive integer ≥2, provided that when one or more raw material Y-th feeding inlet(s) is(are) present, the arrangement of the raw material first feeding inlet(s) and the raw material Y-th feeding inlet(s) make the ratio of the feed amount of the raw material Y-th feeding inlet(s) to the feed amount of the (Y−1)-th raw material feeding inlet(s) be 1:1-10.

3. The fluidized bed reactor according to claim 1, characterized in that at least one catalyst first feeding inlet(s) (24) is arranged on the side wall of reactor between the raw material first distributor (8) and the raw material second distributor (11); and
the raw material first distributor (8) includes a first distributor central region (40) and a first distributor external annular region (42) located on the periphery of the first distributor central region (40), the first distributor external annular region (42) is provided with a first distributor enhancement region (38) corresponding to the radial position of the catalyst first feeding inlet(s) (24), and the first distributor enhancement region (38) has a reduced hole diameter relative to other regions of the raw material first distributor (8), so that the catalyst has a substantially uniform distribution in the radial direction between the raw material first distributor (8) and the raw material second distributor (11).

4. The fluidized bed reactor according to claim 3, characterized in that the first distributor central region (40) is a circle with a radius r, and the first distributor external region (42) is a circular ring with the difference between the outer diameter and the inner diameter being d, wherein r/d=½−⅗, r+d=D, and D is the inner diameter of the fluidized bed reactor; the area of the first distributor enhancement region (38) is set to a ratio of 1/10-½ to the area of the first distributor external region (42).

5. The fluidized bed reactor according to claim 3, characterized in that the region(s) of the raw material first distributor (8) other than the first distributor enhancement region (38) has an opening ratio of 1.5-10%, preferably 2-5%, and a hole diameter of 2-30 mm, preferably the hole diameter difference between each hole does not exceed ±10%; the opening ratio of the first distributor enhancement region (38) is 0.01-1.5% and the hole diameter is 0.1-20 mm, preferably the hole diameter difference between each hole does not exceed ±10%.

6. The fluidized bed reactor according to claim 3, characterized in that the first distributor enhancement region (38) is provided with a plurality of columnar first distributor enhanced nozzles (39), wherein the included angle formed by the center line of the first distributor enhanced nozzle(s) (39) and the horizontal direction is 45°-75°.

7. The fluidized bed reactor according to claim 6, characterized in that the first distributor enhanced nozzle(s) (39) includes an enhanced nozzle inlet (39-1), an enhanced nozzle reducing pipe (39-2), the enhanced nozzle pipe throat (39-3), an enhanced nozzle expansion section (39-4) and an enhanced nozzle outlet (39-5) connected in sequence, wherein the enhanced nozzle inlet (39-1) is connected to the main body of the raw material first distributor (8); wherein,
the included angle range formed by the enhanced nozzle reducing pipe (39-2) and the horizontal direction is 30°-70°, and the included angle range formed by the enhanced nozzle expansion section (39-4) and the horizontal direction is 30°-70°, the ratio of the diameter of the enhanced nozzle pipe throat (39-3) to the diameter of the enhanced nozzle inlet (39-1) is 1:5-20, and the ratio of the length of the enhanced nozzle pipe throat (39-3) to the diameter of the enhanced nozzle pipe throat (39-3) is 5-10:1.

8. The fluidized bed reactor according to claim 1, characterized in that the section of the fluidized bed reactor from the raw material first distributor (8) upward to before diameter reduction has a height h, and the raw material second distributor (11) is arranged at an axial distance of ¼-½h from the raw material first distributor (8).

9. The fluidized bed reactor according to claim 8, characterized in that the opening ratio of the raw material second distributor (11) is 0.05-5%, preferably 3-5%, and the hole diameter is 1-30 mm, preferably the hole diameter difference between each hole does not exceed ±10%.

10. The fluidized bed reactor according to claim 8, characterized in that the raw material second distributor (11) is provided with a second distributor gas main guide pipe (43) extending along the radial direction of the raw material second distributor (11), a plurality of the second distributor gas annulus gap guide pipes (44) arranged sequentially along the radial direction of the raw material second distributor (11), and a second distributor tuyere (45) arranged on the second distributor gas annulus gap guide pipe (44) and a second distributor solid guide groove(s) (46), each of the second distributor gas annulus gap guide pipes (44) is arranged to be distributed in an annular shape around the central region of the raw material second distributor (11), and the second distributor solid guide groove (46) is located between the two adjacent the second distributor gas annulus gap guide pipes (44); wherein the fluidized bed reactor has a raw material second feeding inlet (2), which is in fluid connection with the second distributor gas main guide pipe (43).

11. The fluidized bed reactor according to claim 10, characterized in that the ratio of the width of the second distributor gas annulus gap guide pipe (44) to the width of the second distributor solid guide groove (46) is 1:2-6.

12. The fluidized bed reactor according to claim 1, characterized in that the catalyst distributor main guide pipe (47) passes through the raw material first distributor (8) and the raw material second distributor (11) from bottom to top, the section of the fluidized bed reactor from the raw material first distributor (8) upward to before diameter reduction has a height h, the height of the catalyst distributor main guide pipe (47) upward from the raw material first distributor (8) is h1, then ¼h<h1≤¾h.

13. The fluidized bed reactor according to claim 12, characterized in that the catalyst distributor (16) is a dendritic arrangement scheme, which includes multi-layers of catalyst distribution components (48) distributed along the up and down direction of the catalyst distributor main guide pipe (47), the catalyst distribution components (48) extends in the radial direction, so that the catalyst transported axially along the catalyst distributor main guide pipe (47) can be distributed radially inside the fluidized bed reactor.

14. The fluidized bed reactor according to claim 13, characterized in that the catalyst distribution components (48) include a plurality of catalyst first distribution conduits (49) and a plurality of catalyst second distribution conduits (50), the catalyst first distribution conduits (49) and the catalyst second distribution conduits (50) are distributed circumferentially and staggered along the catalyst distributor main guide pipe (47) and are both connected to the catalyst distributor main guide pipe (47), and the catalyst first distribution conduits (49) and the catalyst second distribution conduits (50) are respectively provided with a plurality of catalyst outlets (51); wherein
the number of the catalyst distribution conduits in each layer of catalyst distribution components (48) is X (X≥2), the circumferential angle spacing of a plurality of catalyst distribution conduits is in 180°/X distribution; and the catalyst outlet (51) has a shape selected from square, circle and polygon.

15. The fluidized bed reactor according to claim 13, characterized in that the number of the catalyst distribution components (48) is preferably M layers (M≥3), and the catalyst distribution components (48) is the 1st layer, the 2nd layer . . . , the M-th layer from top to bottom; wherein the length of the catalyst distribution conduit in the n-th layer catalyst distribution components (48) is (0.7-0.9)ⁿ*D/2, wherein D is the inner diameter of the reactor, and n is the corresponding number of layers.

16. The fluidized bed reactor according to claim 13, characterized in that the catalyst outlets (51) on the catalyst first distribution conduits (49) and the catalyst second distribution conduits (50) are equidistantly distributed.

17. The fluidized bed reactor according to claim 12, characterized in that the catalyst distributor (16) is a main guide pipe arrangement scheme, which includes a catalyst distributor main guide pipe (47) or only consists of the catalyst distributor main guide pipe (47), wherein the catalyst distributor main guide pipe (47) is tubular, with an upper top open; or
the catalyst distributor (16) is an inner baffle arrangement scheme, which includes a catalyst distributor main guide pipe (47) and a catalyst main guide pipe inner baffle (47-2), preferably, its projected area on the horizontal plane is 1/10-¼ of the inner cross section of the catalyst distributor main guide pipe (47); or
the catalyst distributor (16) is a secondary distribution arrangement scheme, which includes a catalyst distributor main guide pipe (47) and 2 or more dispersed secondary distribution guide pipes (47-3) connected to the top of the catalyst distributor main guide pipe (47), preferably its length extending in the radial direction is (0.1-0.9)*D/2, wherein D is the inner diameter of the reactor; or
the catalyst distributor 16 is a spiral guide pipe arrangement scheme, which includes a catalyst distributor main guide pipe (47) and one or more layer(s) of spiral guide pipe (47-4) distributed axially along the catalyst distributor main guide pipe (47), preferably its length extending in the radial direction is (0.5-0.9)ⁿ*D/2, wherein D is the inner diameter of the reactor, and n is the corresponding number of layers; or
the catalyst distributor (16) is an annular guide pipe arrangement scheme, which includes a catalyst distributor main guide pipe (47) and one or more layer(s) of annular guide pipe (47-5) axially distributed along the catalyst distributor main guide pipe (47), preferably the diameter of each layer of annular guide pipe (47-5) is (0.5-0.9)ⁿ*D/2, wherein D is the inner diameter of the reactor, and n is the corresponding number of layers.

18. The fluidized bed reactor according to claim 3, characterized in that, a circulating distribution baffle (34) connected to the inner wall of the fluidized bed reactor is provided above the catalyst first feeding inlet(s) (24).

19. The fluidized bed reactor according to claim 18, characterized in that the ratio of the distance between the circulating distribution baffle (34) and the catalyst first feeding inlet(s) (24) to the hole diameter of the catalyst first feeding inlet(s) (24) is 1-10:1.

20. The fluidized bed reactor according to claim 18, characterized in that the circulating distribution baffle (34) is provided with a plurality of circulating distribution baffle grooves (37), and the included angle (α) formed by the circulating distribution baffle grooves (37) and the horizontal direction is 30°-75°.

21. A device for preparing low-carbon olefin, characterized in that the device includes a fluidized bed reactor (7) according to claim 1, a settler (9), and a regenerator (10), at least one catalyst first feeding inlet(s) (24) for the first feeding of catalyst into the region between the raw material first distributor (8) and the raw material second distributor (11) is provided on the side wall of the reactor, and a catalyst second feeding inlet(s) (27) for the second feeding of catalyst into the distributor main guide pipe (47) is provided at the bottom of the reactor; wherein:
the settler (9) is connected to the upper part of the reaction zone of the fluidized bed reactor (7), and the lower part of the settler (9) is connected to the catalyst first feeding inlet(s) (24) and the regenerator (10) respectively, and the regenerated catalyst outlet of the regenerator (10) is connected to the catalyst second feeding inlet(s) (27).

22. The device according to claim 21, characterized in that the number of the catalyst first feeding inlet(s) (24) is k, k≥2, the included angle between the center lines of each the catalyst first feeding inlet(s) (24) is 360°/k; and preferably k≤12.

23. The device according to claim 22, characterized in that a lower section of settler (17), an upper section of settler (18) located above the lower section of settler (17) and the settler cyclone separator (19) located in the upper section of settler (18) are provided in the settler (9), and the gas outlet of the settler cyclone separator (19) is connected with the product gas outlet (5) of the settler (9), a settler distribution plate (12) is provided at the lower part of the lower section of settler (17), and the lower part of the settler distribution plate (12) is connected to the catalyst first feeding inlet(s) (24) through the circulation pipe(s) (22), and is connected to the regenerator (10) through the stripper (21).

24. The device according to claim 23, characterized in that the settler distribution plate (12) is provided with a settler first distribution plate holes (35) and a settler second distribution plate holes (36), the settler first distribution plate holes (35) and the settler second distribution plate holes(36) are respectively arranged to be annularly distributed around the central region of the settler distribution plate (12), the size ratio of the settler first distribution plate holes(35) to the settler second distribution plate holes(36) is 1-3:4.

25. The device according to claim 21, characterized in that the top of the fluidized bed reactor (7) is provided with a separation riser (15) extending into the settler (9), and a riser baffle (14) located above the outlet of the separation riser (15) is provided in the settler (9).

26. A method for preparing low-carbon olefin in the device of claim 21, comprising:
the gaseous raw material(s) and the catalyst are reacted in the reaction zone of the fluidized bed reactor;
the obtained product and the entrained catalyst are fed into a settler through the top of the reaction zone;
the product and the entrained catalyst are separated in the settler, wherein a part of the catalyst obtained by separation is directly supplied into the dense-phase zone formed between the raw material first distributor (8) and the raw material second distributors (11) through the catalyst first feeding inlet, and another part is fed into the catalyst distribution zone through the catalyst second feeding inlet(s) after being regenerated by the regenerator.

27. The method according to claim 26, characterized in that the linear velocity of the material(s) in the dense-phase zone is 1-10 m/s.

28. The method according to claim 26, characterized in that, in the catalyst obtained by the separation, the mass ratio of the part fed into the dense-phase zone to the part fed into the regenerator is 1:0.2-1.

29. The method according to claim 26, characterized in that, the ratio of the pressure drop produced by the gaseous raw material(s) when passing through the dense-phase zone to the pressure drop produced by the gaseous raw material(s) when passing through the catalyst distribution zone is 1.5-4:1.

30. The method according to claim 26, characterized in that, after the gaseous raw material(s) pass through the raw material first distributor, the included angle formed by the annulus gap space velocity and the horizontal direction is 45°-75°, the ratio between the internal and external porosity fluctuations is 0.9-0.95:1.
